SEQUENCE LISTING (1 sequence)

SEQ ID NO: 1 - protein, 278 aa, Hepatitis C virus
ETHVTGGNAGRTTAGLVGLLTPGAKQNIQLINTNGSWHINSTALNCNESLNTGWLAGLFYQHKFNSSGCPERLASCRRLTDFAQGWGPISYANGSGLDERPYCWHYPPRPCGIVPAKSVCGPVYCFTPSPVVVGTTDRSGAPTYSWGANDTDVFVLNNTRPPLGNWFGCTWMNSTGFTKVCGAPPCVIGGVGNNTLLCPTDCFRKHPEATYSRCGSGPWITPRCMVDYPYRLWHYPCTINYTIFKVRMYVGGVEHRLEAACNWTRGERCDLEDRDRSE

(19) United States
(12) Patent Application Publication
(10) Pub. No.: US 2023/0330218 A1
(43) Pub. Date: Oct. 19, 2023

(54) HEPATITIS C VIRUS MODIFIED E2 GLYCOPROTEIN AND USES THEREOF AS VACCINES

(71) Applicant: Saint Louis University, St. Louis, MO (US)

(72) Inventors: Ranjit RAY, St. Louis, MO (US); Keith MEYER, St. Louis, MO (US); Tapas PATRA, St. Louis, MO (US)

(73) Assignee: Saint Louis University, St. Louis, MO (US)

(21) Appl. No.: 18/160,926

(22) Filed: Jan. 27, 2023

Related U.S. Application Data
(60) Provisional application No. 63/307,910, filed on Feb. 8, 2022.

(51) Int. Cl. A61K 39/29; A61P 31/14

(52) U.S. Cl. CPC A61K 39/29; A61P 31/14; A61K 2039/6018

(57) ABSTRACT

The present disclosure is directed to modified hepatitis virus C (HCV) E2 glycoprotein antigens and their use for treating, preventing or reducing the severity or likelihood of HCV infection.

### PRIORITY CLAIM

This application claims benefit of priority to U.S. Provisional Application Ser. No. 63/307,910, filed Feb. 8, 2022, the entire contents of which are hereby incorporated by reference.

### STATEMENT REGARDING FEDERALLY-FUNDED RESEARCH

This invention was made with government support under grant no. R01DK122401 awarded by National Institutes of Health. The government has certain rights in the invention.

### REFERENCE TO A SEQUENCE LISTING

This application contains a Sequence Listing XML, which has been submitted electronically and is hereby incorporated by reference in its entirety. Said Sequence Listing XML, created on May 31, 2023, is named USTL.P0145US.xml and is 3 kB in size.

### BACKGROUND

### 1. Field of the Disclosure

The present disclosure relates generally to the fields of medicine, infectious disease, and immunology. More particular, the disclosure relates to variants of hepatitis C virus glycoprotein E2 and their use as vaccine antigens in the generation of protective immune responses.

### 2. Background

HCV has evolved mechanisms to evade immune activation and resolution of infection. Several studies of acute HCV infection demonstrate that virus clearance is associated with broad and potent T-cell activation (Park and Rehermann, 2014; Thimme, 2021) and a rapid induction of cross-reactive neutralizing antibody responses (Pestka et al., 2007; Osburn et al., 2014; Keck et al., 2018; Sevvani et al., 2021). A recent report suggests that vaccination of human subjects with a recombinant virus vector incorporating non-structural proteins lowered HCV RNA level, but did not prevent chronic infection (Page et al., 2021). A Phase I vaccine trial of recombinant HCV E1/E2 EnvGPs in healthy human volunteers was conducted (Frey et al., 2010; Ray et al., 2010). Vaccination failed to exhibit a clear dose dependent response to escalated E1/E2 proteins, and only ˜28% vaccinated human sera displayed detectable virus neutralization response.

A Phase I clinical trial with the candidate HCV E1/E2 vaccine, IL-10 secretion by PBMCs was observed, and generation of a variable range of IL-4 secretion was noted (Frey et al., 2010). HCV E2 induces the immune regulatory cytokine, IL-10, and CD163 protein from primary macrophages (Kwon et al., 2019). Further, HCV E2 enhances STAT3 and suppresses STAT1 activation, suggesting macrophage polarization towards the M2 phenotype. HCV E2 inhibits the function of T-, B- and NK-cells by binding with CD81 (Wack et al., 2001; Crotta et al., 2002; Rosa et al., 2005; Crotta et al., 2006). HCV-specific CD4⁺T-cell immune deficiency is believed to be the primary cause of CD8⁺T-cell functional exhaustion (Grakoui et al., 2003; Lauer, 2013).

### SUMMARY

Thus, in accordance with the present disclosure, a method of generating a robust protective immune response in a subject against hepatitis C virus (HCV) infection or at risk of contracting HCV comprising delivering to said subject a polypeptide comprising residues 384 to 661 of HCV E2 glycoprotein with one or more substitutions of L427Y, F442N, Q444T and/or D535A as compared to reference sequence SEQ ID NO: 1 which illustrate residues 384 to 661 of wild-type HCV E2. The only substitution may be L427Y. The only substitution may be F442N. The only substitution may be Q444T. The only substitution may be D535A. The only substitutions may be D535A, F442N, and Q444T The polypeptide may comprise or be limited to 2, 3 or 4 of said substitutions.

Delivering may comprise polypeptide administration, or genetic delivery with an RNA or DNA sequence or vector encoding the polypeptide. The RNA or DNA sequence may be delivered as part of a lipid nanoparticle. The subject may be infected with HCV as determined by diagnostic testing and/or clinical diagnosis. The subject may have been exposed to HCV but is asymptomatic. The subject may be neither infected nor have been exposed to HCV.

In another embodiment, there is a hepatitis C virus (HCV) polypeptide comprising residues 384 to 661 of HCV E2 glycoprotein with one or more substitutions of L427Y, F442N, Q444T and/or D535A as compared to reference sequence SEQ ID NO: 1 which illustrate residues 384 to 661 of wild-type HCV E2. The polypeptide may only have a substitution as compared to reference sequence SEQ ID NO: 1 of L247Y, F442N or Q444T. The polypeptide may only have substitutions as compared to reference sequence SEQ ID NO: 1 of F442N/Q444T. The polypeptide may comprise or be limited to 2, 3 or 4 of said substitutions.

In yet another embodiment, there is provided a vaccine formulation comprising a hepatitis C virus (HCV) peptide or polypeptide comprising residues 384 to 661 of HCV E2 glycoprotein with one or more substitutions of L427Y, F442N, Q444T and/or D535A as compared to reference sequence SEQ ID NO: 1 which illustrate residues 384 to 661 of wild-type HCV E2. The vaccine formulation may be lyophilized.

The use of both the sE1₁₉₃₋₃₅₁ and SE2_{F442NYT} mutant will likely offer a much broader protective immune response. The vaccine formulation may be a liquid formulation comprising said peptide or polypeptide in a pharmaceutically acceptable diluent. The vaccine formulation may further comprise an adjuvant. The vaccine formulation may be sterile.

Also provided is a vaccine formulation comprising an RNA or DNA encoding hepatitis C virus (HCV) peptide or polypeptide comprising residues 384 to 661 of HCV E2 glycoprotein with one or more substitutions of L427Y, F442N, Q444T and/or D535A as compared to reference sequence SEQ ID NO: 1 which illustrate residues 384 to 661 of wild-type HCV E2. The vaccine formulation may be lyophilized. The vaccine formulation may be a liquid formulation comprising said RNA or DNA in a pharmaceutically acceptable diluent. The RNA or DNA may be in a lipid nanoparticle. The vaccine formulation may be sterile.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

It is contemplated that any method or composition described herein can be implemented with respect to any other method or composition described herein. Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the present disclosure. The disclosure may be better understood by reference to one or more of these drawings in combination with the detailed description of specific embodiments presented herein.

FIGS. 1A-C. Schematic presentation of HCV E2 WT ectodomain and the mutants. Amino acid positions of interest are indicated (FIG. 1A). Comparison of CD81 binding with E2 mutant expressed transiently in cell culture supernatant (FIG. 1B) and purified histidine tag recombinant protein (FIG. 1C) are shown. Human recombinant CD81 was coated on ELISA plate and incubated with culture supernatant from HEK293 FS-cells transfected with pcDNA3.1 plasmid containing HCV E2 wild-type or mutant (FIG. 1B) or corresponding purified recombinant protein (FIG. 1C). Culture medium from mock transfected HEK293 FS cells or PBS were used as reagent control for culture supernatant or purified protein, respectively for comparison. The levels of significance are expressed as *=p<0.05 **=p<0.005, ***=p<0.001 and ns=no significance.

FIGS. 2A-D. IL-10 and IL-12 cytokine expression status in mutant E2 protein exposed human and murine macrophages. Human monocyte derived macrophage cell line THP-1 (FIGS. 2A and B) or mouse peritoneum exudate cells (PECs) (FIGS. 2C and D) were treated with 2.5 μg/ml for 24 h and secretory cytokines in the culture supernatant were quantified by ELISA. PECs isolated from 6-8-week-old female BALB/c mice were stimulated with 2.5 μg/ml HCV E2 WT or mutant protein for 24 hrs and secretory cytokines in the culture supernatant were quantified by ELISA. The significance level is expressed as *=p<0.05 **=p<0.005, ***=p<0.001, or ns=not significant.

FIG. 3A-F. Cytokine production from macrophages of healthy donors incubated with purified E2 mutants. Monocyte derived macrophages from human donors (n=6) were incubated with 2.5 μg/ml HCV E2 WT or mutant proteins for 24 h and secretory cytokines in the culture supernatant were quantified by ELISA. The significance level is expressed as *=p<0.05, **=p<0.005, ***=p<0.001, or ns=not significant.

FIG. 4. Effect of E2 stimulation on the expression of co-stimulatory molecules on human macrophage surface. Monocyte derived macrophages from healthy donors (n=4) were stimulated with E2 WT or E2 DM for 24 hrs. in vitro and expression of co-stimulatory molecules on the surface of activated cells were quantified by flowcytometry. All the results were compared with blank controls. The level of significance expressed as *=p<0.05 **=p<0.005, ***=p<0.001 and ns=no significance.

FIGS. 5A-D. The effect of macrophage activation on polarization of CD4⁺T cells toward a Th1 type: CD4⁺T cells were isolated from healthy donors (n=4) and activated with E2 WT or E2 DM for 2 hrs. in the presence of human anti-CD3 (5 μg/ml) and anti-CD28 (1 μg/ml). The expression of T cell activation markers CD69 and CD25 on the surface of CD4⁺T cells were quantified by Flowcytometry (FIGS. 5A and 5B). Monocyte derived macrophages isolated from healthy donors (n=6) were activated with E2 WT or E2 DM protein for 24 hrs and autologous CD4⁺T cells were co-cultured with these activated macrophages for 4 days. Th1 and Th2 polarization were measured by expression of CXCR3 and CCR4, respectively, on the surface of CD4⁺T cells by flowcytometry (FIGS. 5C and 5D). The level of significance expressed as *=p<0.05 **=p<0.005, ***=p<0.001, and ns=no significance.

FIGS. 6A-D. Mouse cytokine response after immunization. Comparison of IL-4, IL-10 (panel A), IL-12 (panel B) and IFN-γ expression quantified by ELISA from sera of unimmunized control, wild-type E2, or mutant E2 vaccinated mice. The level of significance is expressed as *=p<0.05 **=p<0.005, ***=p<0.001, or ns=not significant. FIG. 6C represents data from pooled 5 mouse sera. The IL-4 level shown in FIG. 6D was measured from 2 mouse sera for limited availability.

FIGS. 7A-D. Mouse isotype specific antibody response. Immunized mouse sera (collected after 4 weeks) were analyzed for isotype specific antibody response coating purified E2 antigen on commercially available ELISA plate for isotype antibody analyses. Mouse 1-5 denote wild-type E1-E2 (FIG. 7A) and mouse 6-10 denote E1-E2 double mutant-mRNA-LNP (FIG. 7B) immunized animal sera (FIGS. 7A and 7B). Neutralization of HCVpp was carried out using individual sera from the same immunized animals as described in the above two panels) (FIGS. 7C and 7D).

FIG. 8. Protection of immunized mouse following challenge infection of rVV-expressing HCV E1/E2/NS2ₐₐ₁₃₄₋₉₆₆. Female Balb/c mice (5 in each group) were immunized with vehicle control, E1/E2384-661 (WT) or E1/E2DM (amino acid 442FYQ444 positions are mutated to NYT)-mRNA-LNP. Mouse ovaries were collected after rVV challenge infection, homogenized in medium, freeze-thawed 3 times and centrifuged to pellet cell debris. Clear supernatant was serially 10-fold diluted and plaque assayed on BSC40 cells. Stained plaques were counted, and expressed as pfu/ml. The level of significance is expressed as *=p<0.05 or ns=not significant.

FIGS. 9A-B. Protective responses of HCV sE1/sE2, sE1, sE2 or sE2_{F442NYT} mRNA-LNP immunized mice. (FIG. 9A) Recombinant vaccinia virus titers were determined by plaque forming assay from the ovaries of immunized BALB/c mice after the vaccinia virus (expressing HCV E1-E2-NS2ₐₐ₁₃₄₋₉₆₆) challenge and (FIG. 9B) The levels of IL-2, IFN-γ and granzyme B were quantified by ELISA from ovary homogenates of the recombinant vaccinia challenged mice. The results are presented as the mean with SD. ‘*’, ‘**’, ‘***’ considered statistical significance with p value of <0.05, <0.005, <0.0005, and ‘ns’ denotes as non-significant.

FIGS. 10A-B. Antibody responses of HCV sE1/sE2, sE1, sE2 or sE2_{F442NYT} mRNA-LNP immunized mice. (FIG. 10A) Comparative neutralization of lentivirus derived HCVpp by sera from immunized mice at different dilutions. The IC₅₀ value of each vaccinated group is indicated as the exact dilution of the serum showing 50% neutralizing activity in each vaccinated group of 5 mice with standard errors in vertical lines. (FIG. 10B) Serum antibody reactivity from immunized mice to HCV E1 (aa314-331) and E2 (aa404-421 and aa429-446) specific peptides were determined. The results are presented as the mean with SD. ‘*’, ‘**’ considered statistical significance with p value of <0.05, <0.005 and ‘ns’ denotes as non-significant.

FIGS. 11A-D. Serum cytokine status of HCV mRNA-LNP vaccinated animals. (FIGS. 11A-D) The comparative levels of IL-2, IFN-γ, IL-4 and IL-10 were quantified by ELISA from the serum of HCV sE1/sE2, sE1, sE2 or sE2_{F442NYT} mRNA-LNP immunized mice. The results are presented as the mean with SD. ‘*’, ‘**’, ‘***’ considered statistical significance with p value of <0.05, <0.005, <0.0005, and ‘ns’ denotes as non-significant.

FIGS. 12A-B. Relative serum immunoglobulins in immunized mice are shown. (A) Percentage of total IgG, IgM and IgA were examined evaluated from the serum of immunized mice. (B) Immunized mouse sera were assessed for different IgG isotype specific responses by ELISA. Isotype changes in immunized mice induced by antigens are shown. The results are presented as the mean with SD, and ‘*’ considered statistical significance with p value of <0.05.

FIGS. 13A-C. Protection from HCV sE1/sE2_{F442NYT} mRNA-LNP immunization of mice. (FIG. 13A) HCVpp neutralization by mouse sera immunized with sE2_{F442NYT} alone or together with sE1 vaccine antigen. (FIG. 13B) Result from surrogate vv/HCVE1-E2-NS2₁₃₄₋₉₆₆ or vv/HCVE2-NS2-NS3₃₆₄₋₁₆₁₉ challenge infection is shown as vaccinia virus recovery by plaque assay from clarified ovary homogenate. (FIG. 13C) IL-2, IFN-γ, and granzyme B levels were measured by ELISA from clarified ovary homogenates of immunized mice. The results are presented as the mean with SD. ‘*’, ‘***’ considered statistical significance with p value of <0.05, <0.0005, and ‘ns’ denotes as non-significant.

### DESCRIPTION OF ILLUSTRATIVE EMBODIMENTS

As discussed above, there presently is no effective vaccine for HCV. Because HCV E2 contains multiple epitopes for both T and B cells (Qiu et al., 2008; Naarding et al., 2011), the inventors examined whether impairing the E2-CD81 interaction can promote T-helper cell functions to induce a robust HCV E2 antigen specific immune response. Site-specific mutations in the E2 extracellular loop (ECL) sequence (soluble or sE2) reduced CD81 binding in that was previously identified as an important property when evaluating HCV candidate vaccine potential (Kwon et al., 2019). The altered sE2 double mutant (at amino acid positions 442 and 444) displayed a significant reduction of CD81 binding, while retaining reactivity with antibodies directed to some of the specific antigenic regions. Introduction of the sE2 mutant (F442NYT) induced pro-inflammatory Th1 related cytokines and suppressed the anti-inflammatory response in primary monocyte-derived macrophages and related cell lines. Immunization of Balb/c mice with an E1/sE2 mutant RNA-lipid nanoparticle (LNP) vaccine displayed IgG1 to IgG2a isotype switching, improved HCV pseudotype, virus neutralization, and resistance to challenge infection with a recombinant vaccinia virus expressing HCV E1/E2/NS2₍ₐₐ₁₃₄₋₉₆₆₎ when compared to similar parental E1/E2 vaccination. Thus, the results reported here suggest that reduced HCV sE2_{F442NQT}-CD81 interaction improved immune response, contributing to the development of an HCV vaccination strategy.

These and other aspects of the disclosure are described in detail below.

### I. Hepatitis C Virus

The hepatitis C virus (HCV) is a small (55-65 nm in size), enveloped, positive-sense single-stranded RNA virus of the family Flaviviridae. The hepatitis C virus is the cause of hepatitis C and some cancers such as liver cancer (hepatocellular carcinoma, abbreviated HCC) and lymphomas in humans. HCV belongs to the genus Hepacivirus, a member of the family Flaviviridae. Before 2011, it was the only member of this genus; however, a member of this genus has been discovered in dogs: canine Hepacivirus. There is also at least one virus in this genus that infects horses. Several additional viruses in the genus have been described in bats and rodents.

The HCV particle consists of a lipid membrane envelope that is 55 to 65 nm in diameter. Two viral envelope glycoproteins, E1 and E2, are embedded in the lipid envelope. They take part in viral attachment and entry into the cell. Within the envelope is an icosahedral core that is 33 to 40 nm in diameter. Inside the core is the RNA material of the virus. E1 and E2 are covalently bonded when embedded in the envelope of HCV and are stabilized by disulfide bonds. E2 is globular and seems to protrude 6 nm out from the envelope membrane according to electron microscope images. These glycoproteins play an important role in the interactions hepatitis C has with the immune system. A hypervariable region, the hypervariable region 1 (HVR1) can be found on the E2 glycoprotein. HVR1 is flexible and quite accessible to surrounding molecules. HVR1 helps E2 shield the virus from the immune system. It prevents CD81 from latching onto its respective receptor on the virus. In addition, E2 can shield E1 from the immune system. Although HVR1 is quite variable in amino acid sequence, this region has similar chemical, physical, and conformational characteristics across many E2 glycoproteins.

Hepatitis C virus has a positive sense single-stranded RNA genome. The genome consists of a single open reading frame that is 9,600 nucleotide bases long. This single open reading frame is translated to produce a single protein product, which is then further processed to produce smaller active proteins. This is why on publicly available databases, such as the European Bioinformatics Institute, the viral proteome only consists of 2 proteins. At the 5′ and 3′ ends of the RNA are the untranslated regions (UTR), that are not translated into proteins but are important to translation and replication of the viral RNA. The 5′ UTR has a ribosome binding site or internal ribosome entry site (IRES) that initiates the translation of a very long protein containing about 3,000 amino acids. The core domain of the HCV IRES contains a four-way helical Holliday junction that is integrated within a predicted pseudoknot. The conformation of this core domain constrains the open reading frame's orientation for positioning on the 40S ribosomal subunit. The large pre-protein is later cleaved by cellular and viral proteases into the 10 smaller proteins that allow viral replication within the host cell or assemble into the mature viral particles. Structural proteins made by the hepatitis C virus include Core protein, E1 and E2; nonstructural proteins include NS2, NS3, NS4A, NS4B, NS5A, and NS5B. The proteins of this virus are arranged along the genome in the following order: N terminal-core-envelope (E1)-E2-p7-nonstructural protein 2 (NS2)-NS3-NS4A-NS4B-NS5A-NS5B-C terminal.

The mature nonstructural proteins (NS2 to NS5B) generation relies on the activity of viral proteinases. The NS2/NS3 junction is cleaved by a metal-dependent autocatalytic proteinase encoded within NS2 and the N-terminus of NS3. The remaining cleavages downstream from this site are catalyzed by a serine protease also contained within the N-terminal region of NS3. The core protein has 191 amino acids and can be divided into three domains on the basis of hydrophobicity: domain 1 (residues 1-117) contains mainly basic residues with two short hydrophobic regions; domain 2 (residues 118-174) is less basic and more hydrophobic and its C-terminus is at the end of p21; domain 3 (residues 175-191) is highly hydrophobic and acts as a signal sequence for E1 envelope protein.

Both envelope proteins (E1 and E2) are highly glycosylated and important in cell entry. E1 serves as the fusogenic subunit and E2 acts as the receptor binding protein. E1 has 4-5 N-linked glycans and E2 has 11 N-glycosylation sites.

NS1 (p7) protein is dispensable for viral genome replication but plays a critical role in virus morphogenesis. This protein is a 63 amino acid membrane-spanning protein which locates itself in the endoplasmic reticulum. Cleavage of p7 is mediated by the endoplasmic reticulum's signal peptidases. Two transmembrane domains of p7 are connected by a cytoplasmic loop and are oriented towards the endoplasmic reticulum's lumen. NS2 protein is a 21-23 kiloDalton (kDa) transmembrane protein with protease activity. NS3 is 67 kDa protein whose N-terminal has serine protease activity and whose C-terminal has NTPase/helicase activity. It is located within the endoplasmic reticulum and forms a heterodimeric complex with NS4A, a 54 amino acid membrane protein that acts as a cofactor of the proteinase. NS4B is a small (27 kDa) hydrophobic integral membrane protein with four transmembrane domains. It is located within the endoplasmic reticulum and plays an important role for recruitment of other viral proteins. It induces morphological changes to the endoplasmic reticulum forming a structure termed the membranous web. NS5A is a hydrophilic phosphoprotein which plays an important role in viral replication, modulation of cell signaling pathways and the interferon response. It is known to bind to endoplasmic reticulum anchored human VAP proteins. The NS5B protein (65 kDa) is the viral RNA-dependent RNA polymerase. NS5B has the key function of replicating the HCV's viral RNA by using the viral positive sense RNA strand as its template and catalyzes the polymerization of ribonucleoside triphosphates (rNTP) during RNA replication. Several crystal structures of NS5B polymerase in several crystalline forms have been determined based on the same consensus sequence BK (HCV-BK, genotype 1). The structure can be represented by a right-hand shape with fingers, palm, and thumb. The encircled active site, unique to NS5B, is contained within the palm structure of the protein. Recent studies on NS5B protein genotype 1b strain J4's (HC-J4) structure indicate a presence of an active site where possible control of nucleotide binding occurs and initiation of de novo RNA synthesis. De novo synthesis adds necessary primers for initiation of RNA replication. Current research attempts to bind structures to this active site to alter its functionality to prevent further viral RNA replication. An 11th protein has also been described. This protein is encoded by a +1 frameshift in the capsid gene. It appears to be antigenic, but its function is unknown.

Replication of HCV involves several steps. The virus replicates mainly in the hepatocytes of the liver, where it is estimated that daily each infected cell produces approximately fifty virions (virus particles) with a calculated total of one trillion virions generated. The virus may also replicate in peripheral blood mononuclear cells, potentially accounting for the high levels of immunological disorders found in chronically infected HCV patients. In the liver, the HCV particles are brought into the hepatic sinusoids by blood flow. These sinusoids neighbor hepatocyte cells. HCV is able to pass through the endothelium of the sinusoids and make its way to the basolateral surface of the hepatocyte cells.

HCV has a wide variety of genotypes and mutates rapidly due to a high error rate on the part of the virus' RNA-dependent RNA polymerase. The mutation rate produces so many variants of the virus it is considered a quasispecies rather than a conventional virus species. Entry into host cells occur through complex interactions between virions, especially through their glycoproteins, and cell-surface molecules CD81, LDL receptor, SR-BI, DC-SIGN, Claudin-1, and Occludin.

The envelope of HCV is similar to very low-density lipoproteins (VLDL) and low-density lipoproteins (LDL). Because of this similarity, the virus is thought to be able to associate with apolipoproteins. It could surround itself with lipoproteins, partially covering up E1 and E2. Recent research indicates that these apolipoproteins interact with scavenger receptor B1 (SR-B1). SR-B1 is able to remove lipids from the lipoproteins around the virus to better allow for HVR1 contact. Claudin 1, which is a tight-junction protein, and CD81 link to create a complex, priming them for later HCV infection processes. As the immune system is triggered, macrophages increase the amount of TNF-α around the hepatocytes which are being infected. This triggers the migration of occludin, which is another tight-junction complex, to the basolateral membrane. The HCV particle is ready to enter the cell. These interactions lead to the endocytosis of the viral particle. This process is aided by clathrin proteins.

Once inside an early endosome, the endosome and the viral envelope fuse and the RNA is allowed into the cytoplasm where HCV takes over portions of the intracellular machinery to replicate. The HCV genome is translated to produce a single protein of around 3,011 amino acids. The polyprotein is then proteolytically processed by viral and cellular proteases to produce three structural (virion-associated) and seven nonstructural (NS) proteins. Alternatively, a frameshift may occur in the Core region to produce an alternate reading frame protein (ARFP). HCV encodes two proteases, the NS2 cysteine autoprotease and the NS3-4A serine protease. The NS proteins then recruit the viral genome into an RNA replication complex, which is associated with rearranged cytoplasmic membranes. RNA replication takes place via the viral RNA-dependent RNA polymerase NS5B, which produces a negative strand RNA intermediate. The negative strand RNA then serves as a template for the production of new positive strand viral genomes. Nascent genomes can then be translated, further replicated or packaged within new virus particles.

The virus replicates on intracellular lipid membranes. The endoplasmic reticulum in particular is deformed into uniquely shaped membrane structures termed ‘membranous webs’. These structures can be induced by sole expression of the viral protein NS4B. The core protein associates with lipid droplets and utilizes microtubules and dyneins to alter their location to a perinuclear distribution. Release from the hepatocyte may involve the VLDL secretory pathway. Another hypothesis states that the viral particle may be secreted from the endoplasmic reticulum through the endosomal sorting complex required for transport (ESCRT) pathway. This pathway is normally utilized to bud vesicles out of the cell. The only limitation to this hypothesis is that the pathway is normally used for cellular budding, and it is not known how HCV would commandeer the ESCRT pathway for use with the endoplasmic reticulum.

Hepatitis C virus is predominantly a blood-borne virus, with very low risk of sexual or vertical transmission. Because of this mode of spread the key groups at risk are intravenous drug users (IDUs), recipients of blood products and sometimes patients on haemodialysis. Common setting for transmission of HCV is also intra-hospital (nosocomial) transmission, when practices of hygiene and sterilization are not correctly followed in the clinic. A number of cultural or ritual practices have been proposed as a potential historical mode of spread for HCV, including circumcision, genital mutilation, ritual scarification, traditional tattooing and acupuncture. It has also been argued that given the extremely prolonged periods of persistence of HCV in humans, even very low and undetectable rates of mechanical transmission via biting insects may be sufficient to maintain endemic infection in the tropics, where people receive large number of insect bites.

### II. Vaccines

In one embodiment, the present disclosure provides an immunogenic composition for inducing an immune response against HCV in a subject, for example, in one embodiment, a vaccine. For a composition to be useful as a vaccine, the composition must induce an immune response against the HCV antigen in a cell, tissue or mammal (e.g., a human). In some instances, the vaccine induces a protective immune response in the mammal. As used herein, an “immunogenic composition” comprises an HCV antigen (e.g., a peptide or polypeptide) as disclosed, a nucleic acid encoding such an antigen, a cell expressing or presenting such an antigen or cellular component, a virus expressing or presenting such an antigen or cellular component, or a combination thereof. In particular embodiments, the composition comprises or encodes all or part of any antigens described herein, or an immunogenically functional equivalent thereof. In other embodiments, the composition is in a mixture that comprises an additional immunostimulatory agent or nucleic acids encoding such an agent. Immunostimulatory agents include but are not limited to an additional antigen, an immunomodulator, an antigen presenting cell, lipid nanoparticle, or an adjuvant. In other embodiments, one or more of the additional agent(s) is covalently bonded to the antigen or an immunostimulatory agent, in any combination.

In the context of the present disclosure, the term “vaccine” refers to a composition that induces an immune response upon inoculation into an animal. In some embodiments, the induced immune response provides protective immunity. A vaccine of the present disclosure may vary in its composition of nucleic acid and/or cellular components. In a non-limiting example, a nucleic acid encoding an HCV antigen might also be formulated with an adjuvant. Of course, it will be understood that various compositions described herein may further comprise additional components.

For example, one or more vaccine components may be comprised in a lipid, liposome, or lipid nanoparticle. In another non-limiting example, a vaccine may comprise one or more adjuvants. A vaccine of the present disclosure, and its various components, may be prepared and/or administered by any method disclosed herein or as would be known to one of ordinary skill in the art, in light of the present disclosure.

In various embodiments, the induction of immunity by the expression of the HCV antigen can be detected by observing in vivo or in vitro the response of all or any part of the immune system in the host against the HCV antigen.

For example, a method for detecting the induction of cytotoxic T lymphocytes is well 5 known. A foreign substance that enters the living body is presented to T cells and B cells by the action of antigen presenting cells (APCs). Some T cells that respond to the antigen presented by APC in an antigen specific manner differentiate into cytotoxic T cells (also referred to as cytotoxic T lymphocytes or CTLs) due to stimulation by the antigen. These antigen-stimulated cells then proliferate. This process is referred to herein as “activation” of T cells. Therefore, CTL induction by an epitope of a polypeptide or peptide or combinations thereof can be evaluated by presenting an epitope of a polypeptide or peptide or combinations thereof to a T cell by APC and detecting the induction of CTL. Furthermore, APCs have the effect of activating B cells, CD4+ T cells, CD8+ T cells, macrophages, eosinophils and NK cells.

A method for evaluating the inducing action of CTL using dendritic cells (DCs) as APC is well known in the art. DC is a representative APC having a robust CTL inducing action among APCs. In the methods of the disclosure, the epitope of a polypeptide or peptide or combinations thereof is initially expressed by the DC and then this DC is contacted with T cells. Detection of T cells having cytotoxic effects against the cells of interest after the contact with DC shows that the epitope of a polypeptide or peptide or combinations thereof has an activity of inducing the cytotoxic T cells.

Furthermore, the induced immune response can also be examined by measuring IFN-γ produced and released by CTL in the presence of antigen-presenting cells that carry immobilized peptide or a combination of peptides by visualizing using anti-IFN-γ antibodies, such as an ELISPOT assay.

Apart from DC, peripheral blood mononuclear cells (PBMCs) may also be used as the APC. The induction of CTL is reported to be enhanced by culturing PBMC in the presence of GM-CSF and IL-4. Similarly, CTL has been shown to be induced by culturing PBMC in the presence of keyhole limpet hemocyanin (KLH) and IL-7. The antigens confirmed to possess CTL-inducing activity by these methods are antigens having DC activation effect and subsequent CTL-inducing activity. Furthermore, CTLs that have acquired cytotoxicity due to presentation of the antigen by APC can be also used as vaccines against antigen-associated disorders.

The induction of immunity by expression of HCV antigens can be further confirmed by observing the induction of antibody production against the HCV antigen. For example, when antibodies against an antigen are induced in a laboratory animal immunized with the composition encoding the antigen, and when antigen-associated pathology is suppressed by those antibodies, the composition is determined to induce immunity.

The specificity of the antibody response induced in an animal can include binding to many regions of the delivered antigen, as well as, the induction of neutralization capable antibodies that that prevent infection or reduce disease severity.

The induction of immunity by expression of the HCV antigen can be further confirmed by observing the induction of CD4+ T cells. CD4+ T cells can also lyse target cells, but mainly supply help in the induction of other types of immune responses, including CTL and antibody generation. The type of CD4+ T cell help can be characterized, as Th1, Th2, Th9, Th17, T regulatory (Treg), or T follicular helper (Tfh) cells. Each subtype of CD4+ T cell supplies help to certain types of immune responses. In one embodiment, the composition selectively induces T follicular helper cells, which drive potent antibody responses.

The therapeutic compounds or compositions of the disclosure may be administered prophylactically (i.e., to prevent a disease or disorder) or therapeutically (i.e., to treat a disease or disorder) to subjects suffering from, or at risk of (or susceptible to) developing a disease or disorder. Such subjects may be identified using standard clinical methods. In the context of the present disclosure, prophylactic administration occurs prior to the manifestation of overt clinical symptoms of disease, such that a disease or disorder is prevented or alternatively delayed in its progression. In the context of the field of medicine, the term “prevent” encompasses any activity, which reduces the burden of mortality or morbidity from disease. Prevention can occur at primary, secondary and tertiary prevention levels. While primary prevention avoids the development of a disease, secondary and tertiary levels of prevention encompass activities aimed at preventing the progression of a disease and the emergence of symptoms as well as reducing the negative impact of an already established disease by restoring function and reducing disease-related complications.

A. Antigens

The present disclosure provides a composition that induces an immune response in a subject. In one embodiment, the composition comprises an HCV antigen as disclosed herein. Of particular, the sequences are described in reference to the following:

(SEQ ID NO: 1)
ETHVTGGNAGRTTAGLVGLLTPGAKQNIQLINTNGSWHINSTALNCNESL
NTGWLAGLFYQHKFNSSGCPERLASCRRLTDFAQGWGPISYANGSGLDER
PYCWHYPPRPCGIVPAKSVCGPVYCFTPSPVVVGTTDRSGAPTYSWGAND
TDVFVLNNTRPPLGNWFGCTWMNSTGFTKVCGAPPCVIGGVGNNTLLCPT
DCFRKHPEATYSRCGSGPWITPRCMVDYPYRLWHYPCTINYTIFKVRMYV
GGVEHRLEAACNWTRGERCDLEDRDRSE

This represents residues 384 to 661 of the wild-type HCV E2 sequence. The underlined residues are 427, 442, 444 and 535. In one embodiment, the composition comprises a nucleic acid sequence, which encodes an HCV antigen. For example, in some embodiments, the composition comprises a nucleoside-modified RNA encoding an HCV antigen. In some embodiments, the composition comprises a purified, nucleoside-modified RNA encoding an HCV antigen. The antigen may include, but is not limited to a polypeptide, peptide, protein, virus, or cell that induces an immune response in a subject.

In one embodiment, the antigen comprises a protein comprising a signal peptide (SP) from MHC class II. Other signal peptides that may be used include, but are not limited to, signal sequences derived from IL-2, tPA, mouse and human IgG, and synthetic optimized signal sequences. In one embodiment, the composition comprises a nucleoside-modified RNA comprising a nucleic acid sequence encoding the antigen, wherein the nucleic acid sequence comprises at least one modified nucleoside.

In some embodiments, the HCV antigen comprises an amino acid sequence that is substantially homologous to the amino acid sequence as set forth herein and retains the immunogenic function of the original amino acid sequence. For example, in some embodiments, the amino acid sequence of the HCV antigen has a degree of identity with respect to the original amino acid sequence of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5%.

In one embodiment, the HCV antigen is encoded by a nucleic acid sequence of a nucleic acid molecule. In some embodiments, the nucleic acid sequence comprises DNA, RNA, cDNA, viral DNA, a variant thereof, a fragment thereof, or a combination thereof. In one embodiment, the nucleic acid sequence comprises a modified nucleic acid sequence. For example, in one embodiment the HCV antigen-encoding nucleic acid sequence comprises nucleoside-modified RNA, as described in detail elsewhere herein. In some instances, the nucleic acid sequence comprises include additional sequences that encode linker or tag sequences that are linked to the antigen by a peptide bond.

B. Purification

In certain embodiments, the peptides/polypeptide of the present disclosure may be purified. The term “purified,” as used herein, is intended to refer to a composition, isolatable from other components, wherein the protein is purified to any degree relative to its naturally-obtainable state. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. Other methods for protein purification include, precipitation with ammonium sulfate, PEG, antibodies and the like or by heat denaturation, followed by centrifugation; gel filtration, reverse phase, hydroxylapatite and affinity chromatography; and combinations of such and other techniques.

In purifying an antibody of the present disclosure, it may be desirable to express the polypeptide in a prokaryotic or eukaryotic expression system and extract the protein using denaturing conditions. The polypeptide may be purified from other cellular components using an affinity column, which binds to a tagged portion of the polypeptide. As is generally known in the art, it is believed that the order of conducting the various purification steps may be changed, or that certain steps may be omitted, and still result in a suitable method for the preparation of a substantially purified protein or peptide.

Commonly, complete antibodies are fractionated utilizing agents (i.e., protein A) that bind the Fc portion of the antibody. Alternatively, antigens may be used to simultaneously purify and select appropriate antibodies. Such methods often utilize the selection agent bound to a support, such as a column, filter or bead. The antibodies are bound to a support, contaminants removed (e.g., washed away), and the antibodies released by applying conditions (salt, heat, etc.).

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. Another method for assessing the purity of a fraction is to calculate the specific activity of the fraction, to compare it to the specific activity of the initial extract, and to thus calculate the degree of purity. The actual units used to represent the amount of activity will, of course, be dependent upon the particular assay technique chosen to follow the purification and whether or not the expressed protein or peptide exhibits a detectable activity.

C. Adjuvants and Immunostimulatory Compositions

In one embodiment, the composition comprises an adjuvant. In one embodiment, the composition comprises a nucleic acid molecule encoding an adjuvant. In one embodiment, the adjuvant-encoding nucleic acid molecule is IVT RNA. In one embodiment, the adjuvant-encoding nucleic acid molecule is nucleoside-modified RNA. Exemplary adjuvants and immunostimulatory compositions include, but are not limited to, α-interferon, γ-interferon, platelet derived growth factor (PDGF), TNFα, TNβ, GM-CSF, epidermal growth factor (EGF), cutaneous T cell-attracting chemokine (CTACK), epithelial thymus-expressed chemokine (TECK), mucosae-associated epithelial chemokine (MEC), IL-12, IL-15, MHC, CD80, CD86. Other genes which may be useful adjuvants include those encoding: MCP-I, MIP-Ia, MIP-Ip, IL-8, RANTES, L-selectin, P-selectin, E-selectin, CD34, GlyCAM-1, MadCAM-1, LFA-I, VLA-I, Mac-1, pl50.95, PECAM, ICAM-I, ICAM-2, ICAM-3, CD2, LFA-3, M-CSF, G-CSF, IL-4, mutant forms of IL-18, CD40, CD40L, vascular growth factor, fibroblast growth factor, IL-7, nerve growth factor, vascular endothelial growth factor, Fas, TNF receptor, Fit, Apo-1, p55, WSL-I, DR3, TRAMP, Apo-3, AIR, LARD, NGRF, DR4, DR5, KILLER, TRAIL-R2, TRICK2, DR6, Caspase ICE, Fos, c-jun, Sp-I, Ap-I, Ap-2, p38, p65Rel, MyD88, IRAK, TRAF6, IkB, Inactive NIK, SAP K, SAP-I, JNK, interferon response genes, NFkB, Bax, TRAIL, TRAILrec, TRAILrecDRC5, TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F, TAP 1, TAP2, anti-CTLA4-sc, anti-LAG3-Ig, anti-TIM3-Ig, and functional fragments thereof.

In some embodiments, the composition comprises a lipid nanoparticle, where the lipid nanoparticle acts as an adjuvant.

D. Nucleic Acids

In one embodiment, the disclosure includes a nucleic acid molecule encoding an HCV antigen. In one embodiment, the disclosure includes a nucleoside-modified nucleic acid molecule. In one embodiment, the nucleic acid molecule encodes an HCV antigen. In one embodiment, the nucleic acid molecule encodes a plurality of antigens, including one or more HCV antigens. In some embodiments, the nucleic acid molecule encodes an HCV antigen that induces an adaptive immune response against the HCV antigen. In one embodiment, the disclosure includes a nucleic acid molecule encoding an adjuvant.

The nucleic acid molecule can be made using any methodology in the art, including, but not limited to, in vitro transcription, chemical synthesis, or the like.

The nucleotide sequences encoding an HCV antigen or adjuvant, as described herein, can alternatively comprise sequence variations with respect to the original nucleotide sequences, for example, substitutions, insertions and/or deletions of one or more nucleotides, with the condition that the resulting polynucleotide encodes a polypeptide according to the disclosure. Therefore, the scope of the present disclosure includes nucleotide sequences that are substantially homologous to the nucleotide sequences recited herein and encode an HCV antigen or adjuvant of interest.

As used herein, a nucleotide sequence is “substantially homologous” to any of the nucleotide sequences described herein when its nucleotide sequence has a degree of identity with respect to the original nucleotide sequence at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5%. A nucleotide sequence that is substantially homologous to a nucleotide sequence encoding an antigen can typically be isolated from a producer organism of the antigen based on the information contained in the nucleotide sequence by means of introducing conservative or non-conservative substitutions, for example. Other examples of possible modifications include the insertion of one or more nucleotides in the sequence, the addition of one or more nucleotides in any of the ends of the sequence, or the deletion of one or more nucleotides in any end or inside the sequence. The degree of identity between two polynucleotides is determined using computer algorithms and methods that are widely known for the persons skilled in the art.

Further, the scope of the disclosure includes nucleotide sequences that encode amino acid sequences that are substantially homologous to the amino acid sequences recited herein and preserve the immunogenic function of the original amino acid sequence.

As used herein, an amino acid sequence is “substantially homologous” to any of the amino acid sequences described herein when its amino acid sequence has a degree of identity with respect to the original amino acid sequence of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, of at least 99%, or of at least 99.5%. The identity between two amino acid sequences can be determined by using the BLASTN algorithm (BLAST Manual, Altschul, S., et al., NCBI NLM NIH Bethesda, Md. 20894, Altschul, S., et al., J. Mol. Biol. 215: 403-410, 1990).

In one embodiment, the disclosure relates to a construct, comprising a nucleotide sequence encoding an HCV antigen. In one embodiment, the construct comprises a plurality of nucleotide sequences encoding a plurality of HCV antigens. For example, in some embodiments, the construct encodes 1 or more, 2 or more, 3 or more, or all HCV antigens. In one embodiment, the disclosure relates to a construct, comprising a nucleotide sequence encoding an adjuvant. In one embodiment, the construct comprises a first nucleotide sequence encoding an HCV antigen and a second nucleotide sequence encoding an adjuvant.

In one embodiment, the composition comprises a plurality of constructs, each construct encoding one or more HCV antigens. In some embodiments, the composition comprises 1 or more, 2 or more, 3 or more, 4 or more, 5 or more, 6 or more, 7 or more, 8 or more, 9 or more, 10 or more, 15 or more, or 20 or more constructs. In one embodiment, the composition comprises about 5 to 11 constructs. In one embodiment, the composition comprises a first construct, comprising a nucleotide sequence encoding an HCV antigen; and a second construct, comprising a nucleotide sequence encoding an adjuvant.

In another particular embodiment, the construct is operatively bound to a translational control element. The construct can incorporate an operatively bound regulatory sequence for the expression of the nucleotide sequence of the disclosure, thus forming an expression cassette.

1. Vectors For Delivery of Nucleic Acids

The nucleic acid sequences coding for the HCV antigen or adjuvant can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically.

The nucleic acid can be cloned into a number of types of vectors. For example, the nucleic acid can be cloned into a vector including, but not limited to a plasmid, a phagemid, a phage derivative, an animal virus, a PCR-generated linear DNA sequence, and a cosmid. Vectors of particular interest include expression vectors, replication vectors, probe generation vectors, sequencing vectors and vectors optimized for in vitro transcription.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, carbohydrates, peptides, cationic polymers, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/RNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, MO; dicetyl phosphate (“DCP”) can be obtained from K & K Laboratories (Plainview, NY); cholesterol (“Chol”) can be obtained from Calbiochem-Behring; dimyristyl phosphatidylglycerol (“DMPG”) and other lipids may be obtained from Avanti Polar Lipids, Inc. (Birmingham, AL). Stock solutions of lipids in chloroform or chloroform/methanol can be stored at about −20° C. Chloroform is used as it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as non-uniform aggregates of lipid molecules. Also contemplated are lipofectamine-nucleic acid complexes.

Regardless of the method used to introduce exogenous nucleic acids into a host cell or otherwise expose a cell to a composition of the present disclosure, in order to confirm the presence of the mRNA sequence in the host cell, a variety of assays may be performed. Such assays include, for example, “molecular biological” assays well known to those of skill in the art, such as Northern blotting and RT-PCR; “biochemical” assays, such as detecting the presence or absence of a particular peptide, e.g., by immunogenic means (ELISAs and Western blots) or by assays described herein to identify agents falling within the scope of the disclosure.

2. In Vitro Transcribed RNA In one embodiment, the composition of the disclosure comprises in vitro transcribed (IVT) RNA encoding an HCV antigen. In one embodiment, the composition of the disclosure comprises IVT RNA encoding a plurality of HCV antigens. In one embodiment, the composition of the disclosure comprises IVT RNA encoding an adjuvant. In one embodiment, the composition of the disclosure comprises IVT RNA encoding one or more HCV antigens and one or more adjuvants.

In one embodiment, an IVT RNA can be introduced to a cell as a form of transient transfection. The RNA is produced by in vitro transcription using a plasmid DNA template generated synthetically. DNA of interest from any source can be directly converted by PCR into a template for in vitro mRNA synthesis using appropriate primers and RNA polymerase. The source of the DNA can be, for example, genomic DNA, plasmid DNA, phage DNA, cDNA, synthetic DNA sequence or any other appropriate source of DNA. In one embodiment, the desired template for in vitro transcription is an HCV antigen capable of inducing an adaptive immune response. In one embodiment, the desired template for in vitro transcription is an adjuvant capable of enhancing an adaptive immune response.

In one embodiment, the DNA to be used for PCR contains an open reading frame. The DNA can be from a naturally occurring DNA sequence from the genome of an organism. In one embodiment, the DNA is a full length gene of interest of a portion of a gene. The gene can include some or all of the 5′ and/or 3′ untranslated regions (UTRs). The gene can include exons and introns. In one embodiment, the DNA to be used for PCR is a human gene. In another embodiment, the DNA to be used for PCR is a human gene including the 5′ and 3′ UTRs. In another embodiment, the DNA to be used for PCR is a gene from a pathogenic or commensal organism, including bacteria, viruses, parasites, and fungi. In another embodiment, the DNA to be used for PCR is from a pathogenic or commensal organism, including bacteria, viruses, parasites, and fungi, including the 5′ and 3′ UTRs. The DNA can alternatively be an artificial DNA sequence that is not normally expressed in a naturally occurring organism. An exemplary artificial DNA sequence is one that contains portions of genes that are ligated together to form an open reading frame that encodes a fusion protein. The portions of DNA that are ligated together can be from a single organism or from more than one organism.

Genes that can be used as sources of DNA for PCR include genes that encode polypeptides that induce or enhance an adaptive immune response in an organism. In some instances, the genes are useful for a short-term treatment. In some instances, the genes have limited safety concerns regarding dosage of the expressed gene.

In various embodiments, a plasmid is used to generate a template for in vitro transcription of mRNA, which is used for transfection.

Chemical structures with the ability to promote stability and/or translation efficiency may also be used. In some embodiments, the RNA has 5′ and 3′ UTRs. In one embodiment, the 5′ UTR is between zero and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the gene of interest. Alternatively, UTR sequences that are not endogenous to the gene of interest can be added by incorporating the UTR sequences into the forward and reverse primers or by any other modifications of the template. The use of UTR sequences that are not endogenous to the gene of interest can be useful for modifying the stability and/or translation efficiency of the RNA. For example, it is known that AU-rich elements in 3′ UTR sequences can decrease the stability of mRNA. Therefore, 3′ UTRs can be selected or designed to increase the stability of the transcribed RNA based on properties of UTRs that are well known in the art.

In one embodiment, the 5′ UTR can contain the Kozak sequence of the endogenous gene. Alternatively, when a 5′ UTR that is not endogenous to the gene of interest is being added by PCR as described above, a consensus Kozak sequence can be redesigned by adding the 5′ UTR sequence. Kozak sequences can increase the efficiency of translation of some RNA transcripts but does not appear to be required for all RNAs to enable efficient translation. The requirement for Kozak sequences for many mRNAs is known in the art. In other embodiments the 5′ UTR can be derived from an RNA virus whose RNA genome is stable in cells. In other embodiments various nucleotide analogues can be used in the 3′ or 5′ UTR to impede exonuclease degradation of the mRNA.

To enable synthesis of RNA from a DNA template, a promoter of transcription should be attached to the DNA template upstream of the sequence to be transcribed. When a sequence that functions as a promoter for an RNA polymerase is added to the 5′ end of the forward primer, the RNA polymerase promoter becomes incorporated into the PCR product upstream of the open reading frame that is to be transcribed. In one embodiment, the promoter is a T7 RNA polymerase promoter, as described elsewhere herein. Other useful promoters include, but are not limited to, T3 and SP6 RNA polymerase promoters. Consensus nucleotide sequences for T7, T3 and SP6 promoters are known in the art.

In one embodiment, the mRNA has both a cap on the 5′ end and a 3′ poly(A) tail which determine ribosome binding, initiation of translation and stability of mRNA in the cell. On a circular DNA template, for instance, plasmid DNA, RNA polymerase produces a long concatameric product, which is not suitable for expression in eukaryotic cells. The transcription of plasmid DNA linearized at the end of the 3′ UTR results in normal sized mRNA, which is effective in eukaryotic transfection when it is polyadenylated after transcription.

On a linear DNA template, phage T7 RNA polymerase can extend the 3′ end of the transcript beyond the last base of the template (Schenborn and Mierendorf, Nuc Acids Res., 13:6223-36 (1985); Nacheva and Berzal-Herranz, Eur. J. Biochem., 270:1485-65 (2003)).

The conventional method of integration of polyA/T stretches into a DNA template is molecular cloning. However, polyA/T sequence integrated into plasmid DNA can cause plasmid instability, which can be ameliorated through the use of recombination incompetent bacterial cells for plasmid propagation.

Poly(A) tails of RNAs can be further extended following in vitro transcription with the use of a poly(A) polymerase, such as E. coli polyA polymerase (EPAP) or yeast polyA polymerase. In one embodiment, increasing the length of a poly(A) tail from 100 nucleotides to between 300 and 400 nucleotides results in about a two-fold increase in the translation efficiency of the RNA. Additionally, the attachment of different chemical groups to the 3′ end can increase mRNA stability. Such attachment can contain modified/artificial nucleotides, aptamers and other compounds. For example, ATP analogs can be incorporated into the poly(A) tail using poly(A) polymerase. ATP analogs can further increase the stability of the RNA.

5′ caps also provide stability to mRNA molecules. In one embodiment, RNAs produced by the methods to include a 5′ cap1 structure. Such cap1 structure can be generated using Vaccinia capping enzyme and 2′-O-methyltransferase enzymes (CellScript, Madison, WI). Alternatively, 5′ cap is provided using techniques known in the art and described herein (Cougot, et al., Trends in Biochem. Sci., 29:436-444 (2001); Stepinski, et al., RNA, 7:1468-95 (2001); Elango, et al., Biochim. Biophys. Res. Commun., 330:958-966 (2005).

RNA can be introduced into target cells using any of a number of different methods, for instance, commercially available methods which include, but are not limited to, electroporation (Amaxa Nucleofector-II (Amaxa Biosystems, Cologne, Germany)), (ECM 830 (BTX) (Harvard Instruments, Boston, Mass.) or the Gene Pulser II (BioRad, Denver, Colo.), Multiporator (Eppendort, Hamburg Germany), cationic liposome mediated transfection using lipofection, polymer encapsulation, peptide mediated transfection, or biolistic particle delivery systems such as “gene guns” (see, for example, Nishikawa, et al. Hum Gene Ther., 12(8):861-70 (2001)). In some embodiments RNA of the disclosure is introduced to a cell with a method comprising the use of TransIT®-mRNA transfection Kit (Mirus, Madison WI), which, in some instances, provides high efficiency, low toxicity, transfection.

3. Nucleoside-Modified RNA

In one embodiment, the composition of the present disclosure comprises a nucleoside-modified nucleic acid encoding an HCV antigen as described herein. In one embodiment, the composition of the present disclosure comprises a nucleoside-modified nucleic acid encoding a plurality of antigens, including one or more HCV antigens. In one embodiment, the composition of the present disclosure comprises a nucleoside-modified nucleic acid encoding an adjuvant as described herein. In one embodiment, the composition of the present disclosure comprises a nucleoside-modified nucleic acid encoding one or more HCV antigens and one or more adjuvants.

In one embodiment, the composition of the present disclosure comprises a series of nucleoside-modified nucleic acid encoding one or more HCV antigens that change for each subsequent injection to follow the lineage scheme. For example, in one embodiment, the composition comprises a nucleoside-modified RNA. In one embodiment, the composition comprises a nucleoside-modified mRNA. Nucleoside-modified mRNA has particular advantages over non-modified mRNA, including for example, increased stability, low or absent innate immunogenicity, and enhanced translation. Nucleoside-modified mRNA useful in the present disclosure is further described in U.S. Pat. Nos. 8,278,036, 8,691,966, and 8,835,108, each of which is incorporated by reference herein in its entirety.

In some embodiments, nucleoside-modified mRNA does not activate any pathophysiologic pathways, translates very efficiently and almost immediately following delivery, and serve as templates for continuous protein production in vivo lasting for several days to weeks (Karikó et al., 2008, Mol Ther 16:1833-1840; Karikó et al., 2012, Mol Ther 20:948-953). The amount of mRNA required to exert a physiological effect is small, making it applicable for human therapy. For example, as described herein, nucleoside-modified mRNA encoding an HCV antigen has demonstrated the ability to induce antigen-specific antibody production. For example, in some instances, antigen encoded by nucleoside-modified mRNA induces greater production of antigen-specific antibody production as compared to antigen encoded by non-modified mRNA.

In some instances, expressing a protein by delivering the encoding mRNA has many benefits over methods that use protein, plasmid DNA or viral vectors. During mRNA transfection, the coding sequence of the desired protein is the only substance delivered to cells, thus avoiding all the side effects associated with plasmid backbones, viral genes, and viral proteins. More importantly, unlike DNA- and viral-based vectors, the mRNA does not carry the risk of being incorporated into the genome and protein production starts immediately after mRNA delivery. For example, high levels of circulating proteins have been measured within 15 to 30 minutes of in vivo injection of the encoding mRNA. In some embodiments, using mRNA rather than the protein also has many advantages. Half-lives of proteins in the circulation or in tissues are often short, thus protein treatment would need frequent dosing, while mRNA provides a template for continuous protein production for several days to weeks. Purification of proteins is problematic, and they can contain aggregates and other impurities that cause adverse effects (Kromminga and Schellekens, 2005, Ann NY Acad Sci 1050:257-265).

In some embodiments, the nucleoside-modified RNA comprises the naturally occurring modified-nucleoside pseudouridine. In some embodiments, inclusion of pseudouridine makes the mRNA more stable, non-immunogenic, and highly translatable (Karikó et al., 2008, Mol Ther 16:1833-1840; Anderson et al., 2010, Nucleic Acids Res 38:5884-5892; Anderson et al., 2011, Nucleic Acids Research 39:9329-9338; Karikó et al., 2011, Nucleic Acids Research 39:e142; Karikó et al., 2012, Mol Ther 20:948-953; Karikó et al., 2005, Immunity 23:165-175).

It has been demonstrated that the presence of modified nucleosides, including pseudouridines in RNA suppress their innate immunogenicity (Karikó et al., 2005, Immunity 23:165-175). Further, protein-encoding, in vitro-transcribed RNA containing pseudouridine can be translated more efficiently than RNA containing no or other modified nucleosides (Karikó et al., 2008, Mol Ther 16:1833-1840). Subsequently, it is shown that the presence of pseudouridine improves the stability of RNA (Anderson et al., 2011, Nucleic Acids Research 39:9329-9338) and abates both activation of PKR and inhibition of translation (Anderson et al., 2010, Nucleic Acids Res 38:5884-5892). Similar effects as described for pseudouridine have also been observed for RNA containing 1-methyl-pseudouridine.

In some embodiments, the nucleoside-modified nucleic acid molecule is a purified nucleoside-modified nucleic acid molecule. For example, in some embodiments, the composition is purified to remove double-stranded contaminants. In some instances, a preparative high performance liquid chromatography (HPLC) purification procedure is used to obtain pseudouridine-containing RNA that has superior translational potential and no innate immunogenicity (Karikó et al., 2011, Nucleic Acids Research 39:e142).

Administering HPLC-purified, pseudouridine-containing RNA coding for erythropoietin into mice and macaques resulted in a significant increase of serum EPO levels (Karikó et al., 2012, Mol Ther 20:948-953), thus confirming that pseudouridine-containing mRNA is suitable for in vivo protein therapy. In some embodiments, the nucleoside-modified nucleic acid molecule is purified using non-HPLC methods. In some instances, the nucleoside-modified nucleic acid molecule is purified using chromatography methods, including but not limited to HPLC and fast protein liquid chromatography (FPLC). An exemplary FPLC-based purification procedure is described in Weissman et al., 2013, Methods Mol Biol, 969: 43-54. Exemplary purification procedures are also described in U.S. Patent Application Publication No. US2016/0032316, which is hereby incorporated by reference in its entirety.

The present disclosure encompasses RNA, oligoribonucleotide, and polyribonucleotide molecules comprising pseudouridine or a modified nucleoside. In some embodiments, the composition comprises an isolated nucleic acid encoding an antigen, wherein the nucleic acid comprises a pseudouridine or a modified nucleoside. In some embodiments, the composition comprises a vector, comprising an isolated nucleic acid encoding an antigen, adjuvant, or combination thereof, wherein the nucleic acid comprises a pseudouridine or a modified nucleoside.

In one embodiment, the nucleoside-modified RNA of the disclosure is IVT RNA, as described elsewhere herein. For example, in some embodiments, the nucleoside-modified RNA is synthesized by T7 phage RNA polymerase. In another embodiment, the nucleoside-modified mRNA is synthesized by SP6 phage RNA polymerase. In another embodiment, the nucleoside-modified RNA is synthesized by T3 phage RNA polymerase.

In another embodiment, “exhibits significantly less innate immunogenicity” refers to a detectable decrease in innate immunogenicity. In another embodiment, the term refers to a decrease such that an effective amount of the nucleoside-modified RNA can be administered without triggering a detectable innate immune response. In another embodiment, the term refers to a decrease such that the nucleoside-modified RNA can be repeatedly administered without eliciting an innate immune response sufficient to detectably reduce production of the protein encoded by the modified RNA. In another embodiment, the decrease is such that the nucleoside-modified RNA can be repeatedly administered without eliciting an innate immune response sufficient to eliminate detectable production of the protein encoded by the modified RNA.

4. Lipid Nanoparticle as Delivery Vehicles

In one embodiment, delivery of RNA comprises any suitable delivery method, including exemplary RNA transfection methods described elsewhere herein. In some embodiments, delivery of a RNA to a subject comprises mixing the RNA with a transfection reagent prior to the step of contacting. In another embodiment, a method of present disclosure further comprises administering RNA together with the transfection reagent. In another embodiment, the transfection reagent is a cationic lipid reagent. In another embodiment, the transfection reagent is a cationic polymer reagent. In another embodiment, the transfection reagent is a lipid-based transfection reagent. In another embodiment, the transfection reagent is a protein-based transfection reagent. In another embodiment, the transfection reagent is a carbohydrate-based transfection reagent. In another embodiment, the transfection reagent is a cationic lipid-based transfection reagent. In another embodiment, the transfection reagent is a cationic polymer-based transfection reagent. In another embodiment, the transfection reagent is a polyethyleneimine based transfection reagent. In another embodiment, the transfection reagent is calcium phosphate. In another embodiment, the transfection reagent is Lipofectin®, Lipofectamine®, or TransIT®. In another embodiment, the transfection reagent is any other transfection reagent known in the art.

In another embodiment, the transfection reagent forms a liposome. Liposomes, in another embodiment, increase intracellular stability, increase uptake efficiency and improve biological activity. In another embodiment, liposomes are hollow spherical vesicles composed of lipids arranged in a similar fashion as those lipids, which make up the cell membrane. They have, in another embodiment, an internal aqueous space for entrapping water-soluble compounds and range in size from 0.05 to several microns in diameter. In another embodiment, liposomes can deliver RNA to cells in a biologically active form.

In one embodiment, the composition comprises a lipid nanoparticle (LNP) and one or more nucleic acid molecules described herein. For example, in one embodiment, the composition comprises an LNP and one or more RNA molecules encoding one or more antigens, adjuvants, or a combination thereof. The term “lipid nanoparticle” refers to a particle having at least one dimension on the order of nanometers (e.g., 1-1,000 nm), which includes one or more lipids. In some embodiments, lipid nanoparticles are included in a formulation comprising a RNA as described herein. In some embodiments, such lipid nanoparticles comprise a cationic lipid and one or more excipient selected from neutral lipids, charged lipids, steroids and polymer conjugated lipids (e.g., a pegylated lipid such as a pegylated lipid of structure (IV), such as compound Iva). In some embodiments, the RNA is encapsulated in the lipid portion of the lipid nanoparticle or an aqueous space enveloped by some or all of the lipid portion of the lipid nanoparticle, thereby protecting it from enzymatic degradation or other undesirable effects induced by the mechanisms of the host organism or cells, e.g., an adverse immune response.

In various embodiments, the lipid nanoparticles have a mean diameter of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm, and are substantially non-toxic. In some embodiments, the nucleoside-modified RNA, when present in the lipid nanoparticles, is resistant in aqueous solution to degradation with a nuclease.

The LNP may comprise any lipid capable of forming a particle to which the one or more nucleic acid molecules are attached, or in which the one or more nucleic acid molecules are encapsulated. The term “lipid” refers to a group of organic compounds that are derivatives of fatty acids (e.g., esters) and are generally characterized by being insoluble in water but soluble in many organic solvents. Lipids are usually divided in at least three classes: (1) “simple lipids” which include fats and oils as well as waxes; (2) “compound lipids” which include phospholipids and glycolipids; and (3) “derived lipids” such as steroids.

In one embodiment, the LNP comprises one or more cationic lipids, and one or more stabilizing lipids. Stabilizing lipids include neutral lipids and pegylated lipids.

In one embodiment, the LNP comprises a cationic lipid. As used herein, the term “cationic lipid” refers to a lipid that is cationic or becomes cationic (protonated) as the pH is lowered below the pK of the ionizable group of the lipid, but is progressively more neutral at higher pH values. At pH values below the pK, the lipid is then able to associate with negatively charged nucleic acids. In some embodiments, the cationic lipid comprises a zwitterionic lipid that assumes a positive charge on pH decrease.

In some embodiments, the cationic lipid comprises any of a number of lipid species which carry a net positive charge at a selective pH, such as physiological pH. Such lipids include, but are not limited to, N,N-dioleyl-N,N-dimethylammonium chloride (DODAC); N-(2,3-dioleyloxy)propyl)-N,N,N-trimethylammonium chloride (DOTMA); N,N-distearyl-N,N-dimethylammonium bromide (DDAB); N-(2,3-dioleoyloxy)propyl)-N,N,N-trimethylammonium chloride (DOTAP); 3-(N-(N′,N′-dimethylaminoethane)-carbamoyl)cholesterol (DC-Chol), N-(1-(2,3 -dioleoyloxy)propyl)-N-2-(sperminecarboxamido)ethyl)-N,N-dimethylammonium trifluoracetate (DOSPA), dioctadecylamidoglycyl carboxyspermine (DOGS), 1,2-dioleoyl-3-dimethylammonium propane (DODAP), N,N-dimethyl-2,3-dioleoyloxy)propylamine (DODMA), and N-(1,2-dimyristyloxyprop-3-yl)-N,N-dimethyl-N-hydroxyethyl ammonium bromide (DMRIE).

Additionally, a number of commercial preparations of cationic lipids are available which can be used in the present disclosure. These include, for example, LIPOFECTIN® (commercially available cationic liposomes comprising DOTMA and 1,2-dioleoyl-sn-3-phosphoethanolamine (DOPE), from GIBCO/BRL, Grand Island, N.Y.); LIPOFECTAMINE® (commercially available cationic liposomes comprising N-(1-(2,3-dioleyloxy)propyl)-N-(2-(sperminecarboxamido)ethyl)-N,N-dimethylammonium trifluoroacetate (DOSPA) and (DOPE), from GIBCO/BRL); and TRANSFECTAM® (commercially available cationic lipids comprising dioctadecylamidoglycyl carboxyspermine (DOGS) in ethanol from Promega Corp., Madison, Wis.). The following lipids are cationic and have a positive charge at below physiological pH: DODAP, DODMA, DMDMA, 1,2-dilinoleyloxy-N,N-dimethylaminopropane (DLinDMA), 1,2-dilinolenyloxy-N,N-dimethylaminopropane (DLenDMA).

In one embodiment, the cationic lipid is an amino lipid. Suitable amino lipids useful in the disclosure include those described in WO 2012/016184, incorporated herein by reference in its entirety. Representative amino lipids include, but are not limited to, 1,2-dilinoleyoxy-3-(dimethylamino)acetoxypropane (DLin-DAC), 1,2-dilinoleyoxy-3-morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3-dimethylaminopropane (DLin-2-DMAP), 1,2-dilinoleyloxy-3-trimethylaminopropane chloride salt (DLin-TMA.Cl), 1,2-dilinoleoyl-3-trimethylaminopropane chloride salt (DLin-TAP.Cl), 1,2-dilinoleyloxy-3-(N-methylpiperazino)propane (DLin-MPZ), 3-(N,Ndilinoleylamino)-1,2-propanediol (DLinAP), 3-(N,N-dioleylamino)-1,2-propanediol (DOAP), 1,2-dilinoleyloxo-3-(2-N,N-dimethylamino)ethoxypropane (DLin-EG-DMA), and 2,2-dilinoleyl-4-dimethylaminomethyl-[1,3]-dioxolane (DLin-K-DMA).

In some embodiments, the LNP comprises one or more additional lipids which stabilize the formation of particles during their formation. Suitable stabilizing lipids include neutral lipids and anionic lipids. The term “neutral lipid” refers to any one of a number of lipid species that exist in either an uncharged or neutral zwitterionic form at physiological pH. Representative neutral lipids include diacylphosphatidylcholines, diacylphosphatidylethanolamines, ceramides, sphingomyelins, dihydro sphingomyelins, cephalins, and cerebrosides. Exemplary neutral lipids include, for example, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidylethanolamine (DSPE), 16-O-monomethyl PE, 16-O-dimethyl PE, 18-1-trans PE, 1-stearioyl-2-oleoylphosphatidyethanol amine (SOPE), and 1,2-dielaidoyl-sn-glycero-3-phophoethanolamine (transDOPE). In one embodiment, the neutral lipid is 1,2-distearoyl-sn-glycero-3-phosphocholine (DSPC).

In some embodiments, the LNPs comprise a neutral lipid selected from DSPC, DPPC, DMPC, DOPC, POPC, DOPE and SM. In various embodiments, the molar ratio of the cationic lipid (e.g., lipid of Formula (I)) to the neutral lipid ranges from about 2:1 to about 8:1. In various embodiments, the LNPs further comprise a steroid or steroid analogue.

In some embodiments, the steroid or steroid analogue is cholesterol. In some of these embodiments, the molar ratio of the cationic lipid to cholesterol ranges from about 2:1 to 1:1.

The term “anionic lipid” refers to any lipid that is negatively charged at physiological pH. These lipids include phosphatidylglycerol, cardiolipin, diacylphosphatidylserine, diacylphosphatidic acid, N-dodecanoylphosphatidylethanolamines, N-succinylphosphatidylethanolamines, N-glutarylphosphatidylethanolamines, lysylphosphatidylglycerols, palmitoyloleyolphosphatidylglycerol (POPG), and other anionic modifying groups joined to neutral lipids.

In some embodiments, the LNP comprises glycolipids (e.g., monosialoganglioside GM1). In some embodiments, the LNP comprises a sterol, such as cholesterol.

In some embodiments, the LNPs comprise a polymer conjugated lipid. The term “polymer conjugated lipid” refers to a molecule comprising both a lipid portion and a polymer portion. An example of a polymer conjugated lipid is a pegylated lipid. The term “pegylated lipid” refers to a molecule comprising both a lipid portion and a polyethylene glycol portion. Pegylated lipids are known in the art and include 1-(monomethoxy-polyethyleneglycol)-2,3-dimyristoylglycerol (PEG-s-DMG) and the like.

In some embodiments, the LNP comprises an additional, stabilizing lipid which is a polyethylene glycol-lipid (pegylated lipid). Suitable polyethylene glycol-lipids include PEG-modified phosphatidylethanolamine, PEG-modified phosphatidic acid, PEG-modified ceramides (e.g., PEG-CerC14 or PEG-CerC20), PEG-modified dialkylamines, PEG-modified diacylglycerols, PEG-modified dialkylglycerols. Representative polyethylene glycol-lipids include PEG-c-DOMG, PEG-c-DMA, and PEG-s-DMG. In one embodiment, the polyethylene glycol-lipid is N-[(methoxy poly(ethylene glycol)2000)carbamyl]-1,2-dimyristyloxlpropyl-3-amine (PEG-c-DMA). In one embodiment, the polyethylene glycol-lipid is PEG-c-DOMG). In other embodiments, the LNPs comprise a pegylated diacylglycerol (PEG-DAG) such as 1-(monomethoxy-polyethyleneglycol)-2,3-dimyristoylglycerol (PEG-DMG), a pegylated phosphatidylethanoloamine (PEG-PE), a PEG succinate diacylglycerol (PEG-S-DAG), a pegylated ceramide (PEG-5 cer), or a PEG dialkoxypropylcarbamate. In various embodiments, the molar ratio of the cationic lipid to the pegylated lipid ranges from about 100:1 to about 25:1.

In some embodiments, the LNPs comprise a pegylated lipid. Other exemplary LNPs and their manufacture are described in the art, for example in U.S. Patent Application Publication No. US20120276209, Semple et al., 2010, Nat Biotechnol., 28(2):172-176; Akinc et al., 2010, Mol Ther., 18(7): 1357-1364; Basha et al., 2011, Mol Ther, 19(12): 2186-2200; Leung et al., 2012, J Phys Chem C Nanomater Interfaces, 116(34): 18440-18450; Lee et al., 2012, Int J Cancer., 131(5): E781-90; Belliveau et al., 2012, Mol Ther nucleic Acids, 1: e37; Jayaraman et al., 2012, Angew Chem Int Ed Engl., 51(34): 8529-8533; Mui et al., 2013, Mol Ther Nucleic Acids. 2, e139; Maier et al., 2013, Mol Ther., 21(8): 1570-1578; and Tam et al., 2013, Nanomedicine, 9(5): 665-74, each of which are incorporated by reference in their entirety.

D. Formulations

The formulations of the pharmaceutical compositions described herein may be prepared by any method known or hereafter developed in the art of pharmaceutical formulation and vaccinology. In general, such preparatory methods include the step of bringing the active ingredient into association with a carrier or one or more other accessory ingredients, and then, if necessary or desirable, shaping or packaging the product into a desired single- or multi-dose unit.

Although the description of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the disclosure is contemplated include, but are not limited to, humans and other primates, mammals including commercially relevant mammals such as non-human primates, cattle, pigs, horses, sheep, cats, and dogs.

Pharmaceutical compositions that are useful in the methods of the disclosure may be prepared, packaged, or sold in formulations suitable for ophthalmic, oral, rectal, vaginal, parenteral, topical, pulmonary, intranasal, buccal, intravenous, intracerebroventricular, intradermal, intramuscular, or another route of administration.

Other contemplated formulations include projected nanoparticles, liposomal preparations, resealed erythrocytes containing the active ingredient, and immunogenic-based formulations.

A pharmaceutical composition of the disclosure may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient, which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the disclosure will vary, depending upon the identity, size, and condition of the subject treated and further depending upon the route by which the composition is to be administered. By way of example, the composition may comprise between 0.1% and 100% (w/w) active ingredient. In addition to the active ingredient, a pharmaceutical composition of the disclosure may further comprise one or more additional pharmaceutically active agents.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intraocular, intravitreal, subcutaneous, intraperitoneal, intramuscular, intradermal, intrasternal injection, intravenous, intracerebroventricular and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems.

Compositions for sustained release or implantation may comprise pharmaceutically acceptable polymeric or hydrophobic materials such as an emulsion, an ion exchange resin, a sparingly soluble polymer, or a sparingly soluble salt.

A pharmaceutical composition of the disclosure may be prepared, packaged, or sold in a formulation suitable for pulmonary administration via the buccal cavity. Such a formulation may comprise dry particles which comprise the active ingredient and which have a diameter in the range from about 0.5 to about 7 nanometers. In some embodiments, the formulation may comprise dry particles which comprise the active ingredient and which have a diameter in the range from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. In some embodiments, such powders comprise particles wherein at least 98% of the particles by weight have a diameter greater than 0.5 nanometers and at least 95% of the particles by number have a diameter less than 7 nanometers. In some embodiments, at least 95% of the particles by weight have a diameter greater than 1 nanometer and at least 90% of the particles by number have a diameter less than 6 nanometers. In some embodiments, dry powder compositions include a solid fine powder diluent such as sugar and are conveniently provided in a unit dose form.

Low boiling propellants generally include liquid propellants having a boiling point of below 65° F. at atmospheric pressure. Generally, the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (in some instances having a particle size of the same order as particles comprising the active ingredient).

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations that are useful include those that comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the disclosure are known in the art and described, for example in Remington's Pharmaceutical Sciences (1985, Genaro, ed., Mack Publishing Co., Easton, PA), which is incorporated herein by reference.

### III. Treatment Methods

The present disclosure provides methods of inducing an adaptive immune response against HCV in a subject comprising administering an effective amount of a composition comprising one or more isolated nucleic acids encoding one or more HCV antigens as disclosed herein.

In one embodiment, the method provides immunity in the subject to HCV, HCV infection, or to a disease or disorder associated with HCV. The present disclosure thus provides a method of treating, reducing severity, limiting, reducing the likelihood or preventing of the infection, disease, or disorder associated with HCV.

In one embodiment, the composition is administered to a subject having an infection, disease, or disorder associated with HCV. In one embodiment, the composition is administered to a subject at risk for developing the infection, disease, or disorder associated with HCV. For example, the composition may be administered to a subject who is at risk for being in contact with HCV. In one embodiment, the composition is administered to a subject who lives in, traveled to, or is expected to travel to a geographic region in which HCV is prevalent. In one embodiment, the composition is administered to a subject who is in contact with or expected to be in contact with another person who lives in, traveled to, or is expected to travel to a geographic region in which HCV is prevalent. In one embodiment, the composition is administered to a subject who has knowingly been exposed to HCV through their occupation, sexual, or other contact.

In one embodiment, the method comprises administering a composition comprising one or more nucleic acid molecules encoding one or more HCV antigens. In one embodiment, the method comprises administering a composition comprising a first nucleic acid molecule encoding one or more HCV antigens and a second nucleic acid molecule encoding one or more HCV antigens. In one embodiment, the method comprises administering a composition comprising a one or more nucleic acid molecules encoding a plurality of lineage HCV antigens described herein.

In one embodiment, the method comprises administering one or more compositions, each composition comprising one or more nucleic acid molecules encoding one or more HCV antigens. In one embodiment, the method comprises administering a first composition comprising one or more nucleic acid molecules encoding one or more HCV antigens and administering a second composition comprising one or more nucleic acid molecules encoding one or more HCV antigens. In one embodiment, the method comprises administering a plurality of compositions, each composition comprising one or more nucleic acid molecules encoding one or more lineage HCV antigens described herein. In some embodiments, the method comprises a staggered administration of the plurality of compositions.

In some embodiments, the method comprises administering to subject a plurality of nucleic acid molecules encoding a plurality of HCV antigens, adjuvants, or a combination thereof.

In some embodiments, the method of the disclosure allows for sustained expression of the HCV antigen or adjuvant, described herein, for at least several days following administration. In some embodiments, the method of the disclosure allows for sustained expression of the HCV antigen or adjuvant, described herein, for at least 2 weeks following administration. In some embodiments, the method of the disclosure allows for sustained expression of the HCV antigen or adjuvant, described herein, for at least 1 month following administration. However, the method, in some embodiments, also provides for transient expression, as in some embodiments, the nucleic acid is not integrated into the subject genome.

In some embodiments, the method comprises administering RNA, which provides stable expression of the HCV antigen or adjuvant described herein. In some embodiments, administration of RNA results in little to no innate immune response, while inducing an effective adaptive immune response.

In some embodiments, the method provides sustained protection against HCV. For example, in some embodiments, the method provides sustained protection against HCV for more than 2 weeks. In some embodiments, the method provides sustained protection against HCV for 1 month or more. In some embodiments, the method provides sustained protection against HCV for 2 months or more. In some embodiments, the method provides sustained protection against HCV for 3 months or more. In some embodiments, the method provides sustained protection against HCV for 4 months or more. In some embodiments, the method provides sustained protection against HCV for 5 months or more. In some embodiments, the method provides sustained protection against HCV for 6 months or more. In some embodiments, the method provides sustained protection against HCV for 1 year or more. In one embodiment, a single immunization of the composition induces a sustained protection against HCV for 1 month or more, 2 months or more, 3 months or more, 4 months or more, 5 months or more, 6 months or more, or 1 year or more.

Administration of the compositions of the disclosure in a method of treatment can be achieved in a number of different ways, using methods known in the art. In one embodiment, the method of the disclosure comprises systemic administration of the subject, including for example enteral or parenteral administration. In some embodiments, the method comprises intradermal delivery of the composition. In another embodiment, the method comprises intravenous delivery of the composition. In some embodiments, the method comprises intramuscular delivery of the composition. In one embodiment, the method comprises subcutaneous delivery of the composition. In one embodiment, the method comprises inhalation of the composition. In one embodiment, the method comprises intranasal delivery of the composition.

It will be appreciated that the composition of the disclosure may be administered to a subject either alone, or in conjunction with another agent. The therapeutic and prophylactic methods of the disclosure thus encompass the use of pharmaceutical compositions encoding an HCV antigen, adjuvant, or a combination thereof, described herein to practice the methods of the disclosure. The pharmaceutical compositions useful for practicing the disclosure may be administered to deliver a dose of from 1 ng/kg/day and 100 mg/kg/day. In one embodiment, the disclosure envisions administration of a dose, which results in a concentration of the compound of the present disclosure from 10 nM and 10 μM in a mammal. Typically, dosages which may be administered in a method of the disclosure to a mammal, such as a human, range in amount from 0.01 μg to about 50 mg per kilogram of body weight of the mammal, while the precise dosage administered will vary depending upon any number of factors, including but not limited to, the type of mammal and type of disease state being treated, the age of the mammal and the route of administration. In some embodiments, the dosage of the compound will vary from about 0.1 μg to about 10 mg per kilogram of body weight of the mammal. In some embodiments, the dosage will vary from about 1 μg to about 1 mg per kilogram of body weight of the mammal.

The composition may be administered to a mammal as frequently as several times daily, or it may be administered less frequently, such as once a day, once a week, once every two weeks, once a month, or even less frequently, such as once every several months, several years, or even less frequently, such as every 10-20 years, 15-30 years, or even less frequently, such as every 50-100 years. The frequency of the dose will be readily apparent to the skilled artisan and will depend upon any number of factors, such as, but not limited to, the type and severity of the disease being treated, the type and age of the mammal, etc.

In some embodiments, administration of an immunogenic composition or vaccine of the present disclosure may be performed by single administration or boosted by multiple administrations.

In one embodiment, the disclosure includes a method comprising administering one or more compositions encoding one or more HCV antigens or adjuvants described herein. In some embodiments, the method has an additive effect, wherein the overall effect of the administering the combination is approximately equal to the sum of the effects of administering each HCV antigen or adjuvant. In other embodiments, the method has a synergistic effect, wherein the overall effect of administering the combination is greater than the sum of the effects of administering each HCV antigen or adjuvant.

### IV. EXAMPLES

The following examples are included to demonstrate preferred embodiments. It should be appreciated by those of skill in the art that the techniques disclosed in the examples that follow represent techniques discovered by the inventors to function well in the practice of embodiments, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1

### Materials and Methods

Cells. Human embryonic kidney (HEK) 293T, human monocytic derived THP-1 cell line, human monocyte derived primary macrophages, and murine primary peritoneal macrophages were used. HEK293T cells were maintained in DMEM containing penicillin/streptomycin, 10% fetal calf serum (FCS), 1% L-glutamine and 1% non-essential amino acids. THP-1 cells were maintained in RMPI medium containing 1% penicillin-streptomycin supplemented with 10% FCS, 1% L-glutamine, 25 mM HEPES and 12.5 nM PMA. Primary macrophages were maintained in RPMI containing penicillin/streptomycin supplemented with 10% FCS and 1% L-glutamine.

Site-specific mutagenesis of E2: HCV (H77c genotype 1a) E2 sequence corresponding to 383-661 amino acids cloned into the pcDNA 3.1 vector (gift from Heidi E. Drummer) was isolated from transformed E. coli and used as a template for generation of E2 mutants by a QuikChange Lightning Site Directed Mutagenesis Kit (Agilent Technology) following the supplier's procedure. The inventors chose to express E2 as C-terminal truncations in mammalian cells, since soluble and correctly folded E2 can be obtained by deleting the transmembrane domain as previously reported (Michalak et al., 1997; Flint et al., 1999; Flint et al., 2000; Roccasecca, et al., 2003).

sE2 single mutant (SM) was generated by replacing a non-polar hydrophobic leucine (L) amino acid residue at position 427 to a polar and neutral amino acid tyrosine (L427Y). sE2 double mutant (DM) was generated on the same sE2 harboring single mutant sequence by replacing two amino acids. One replacement was done at position 442 with a non-polar and hydrophobic amino acid phenyl alanine (F) to a polar and hydrophilic amino acid asparagine (F442N) and another mutation at position 444 of a polar and hydrophilic amino acid glutamine (Q) with a neutral and polar amino acid threonine (Q444T). Thus, the terminology single mutant (SM), or double mutant (DM) are used in the text to refer to mutations at L427Y, and F442NYT, respectively. These positions were chosen with referral to previous experimental data (Tzarum et al., 2016) and were tested against a known E2 specific human monoclonal antibodies. The following primers were custom made (IDT) for site specific mutagenesis and generated E2 ectodomain mutants:

(1) Single mutant L427Y
forward primer:
5′-gcacatcaatagcacggcctataactgcaatgaaagccttaa-3′,
reverse primer:
5′-ttaaggctttcattgcagttataggccgtgctattgatgtgc-3′.
(2) Double mutant F442N/Q444T
forward primer:
5′-accggctggttagcagggctcaactatacgcacaaat-3′,
reverse primer:
5′-atttgtgcgtatagttgagccctgctaaccagccggt-3′.

The following PCR conditions were used for PCR amplification of mutant DNAs. Initial denaturation at 95° C. for 2 min 1 cycle, DNA amplification denaturation at 95° C. 20 sec, primer annealing at 60° C. for 10 seconds, extension 68° C. for 3.2 min total 20 cycles and final extension at 68° C. for 5 min. The PCR product was transformed into E. coli XL10 Gold competent cells and colonies were isolated from growth on ampicillin and tetracycline containing LB agar plates. Mutant clones were confirmed by plasmid isolation and DNA sequencing.

Cell transfection, protein expression and purification: HEK293T cells were maintained in Dulbecco's minimal essential medium (DMEM) supplemented with 10% fetal calf serum, 1% penicillin-streptomycin, 1% essential amino acids and 2 mM L-glutamine. Cells (5×10⁵) were seeded on a 6 well plate and incubated at 37° C. in a 5% CO₂ overnight. The plasmid DNA isolated from clones were used for transfection using Lipofectamine™ 3000 Transfection Reagent (ThermoFisher Scientific) following supplier's instructions. Culture supernatant was harvested from plasmid transfected cells after 48 h. The culture supernatant was centrifuged at 3,000 rpm for 10 min at 4° C., the supernatant was passed through a 0.45 μm filter (Nalgene) and stored at −20° C. until purification. The recombinant E2 wild-type or mutant protein containing a hexa-histidine tail was purified by passing through the nickel-NTA-affinity Column (HisPur™ Ni-NTA Spin Columns, ThermoScientific) following supplier's instruction. The purified protein was verified by Coomassie Blue staining and western blot analysis using anti-His antibody (SinoBiologicals).

E2-CD81 binding assay: Recombinant human CD81 protein (R &D Systems) was coated (4 μg/ml) on ELISA plate (Thermoscientific) and incubated overnight at 4° C. Next day, the plate was washed with PBS containing 0.05%Tween-20. The wells were blocked with buffer containing 3% BSA. The plate was washed with wash buffer and 100 or 200 μl HEK293 transfected culture supernatant or 1.25, 2.5 and 5.0 μg/ml purified protein (in triplicates) were added to the wells and incubated at room temperature for 2-3 h. The plate was washed with buffer and 100 μl HRP-conjugated anti-his antibody (1:500 dilution) (SinoBiologicals) was added to each well and incubated at room temperature for 1 h. The plate was washed and TMB substrate was added and the reaction was stopped and read at 450 nm using a microplate reader.

Stimulation of peripheral blood mononuclear cells (PBMC): Fresh blood was collected from healthy donors in a sodium heparin vial and PBMCs were isolated by Ficoll-paque™ PLUS (GE healthcare) density gradient centrifugation as previously described (Kwon et al., 2019). Cells (1×10⁶)/well were seeded on plate and incubated overnight at 37° C. in a CO₂ incubator. Next day, non-adherent cells were removed by washing twice gently and adherent cells were incubated a further 5 days for maturation of monocytes to macrophages. On day 5 macrophages were stimulated with 2.5 μg/ml E2 WT or mutant protein for 24 h at 37° C. Culture supernatants were collected in a fresh, sterile Eppendorf tubes, and centrifuged at 3,000 rpm for 10 min at 18-20° C. Supernatants were transferred to a new sterile tube and stored at −20° C. Adherent cells were lysed with 100 μl 2×SDS loading buffer and stored at −80° C.

Cytokine quantification: Secretory cytokines present in the cell culture supernatant after 24 h were quantified using commercially available capture and matched detection pair antibodies for IL-10, IL-12, IL-6, IFN-γ and TNF-α cytokine kit (SinoBiologicals). IL-4 cytokine was quantified using a kit (Thermoscientific) following supplier's protocol.

Macrophage activation marker analysis: Monocyte derived macrophages were cultured as mentioned above and stimulated with 2.5 μg/ml E2 WT or E2 DM for 24 hrs at 37° C. in a CO₂ incubator. Culture supernatant was removed and 1 ml ice-cold PBS containing 5 mM EDTA was added to each well and incubated on ice for 10-15 min. Cells were collected with gentle pipetting in 1 ml warm PBS containing 5 mM EDTA. Cells were centrifuged at 3000 rpm at 18-20 ° C. for 10 min and suspended in 100 μl (1×10⁶ cell density) flowcytometry stain buffer (PBS containing 10% FBS and 0.1% NaN₃, pH 7.2). Cells were incubated with CD80-FITC, CD86-PE, HLA-DR APC and CD40-PE (Biolegend) antibodies for staining and FACS analysis.

Macrophage/T-cell co-culture and Th1 and Th2 marker analyses: PBMCs isolated from healthy donors (n=6) were used for CD4⁺T cell isolation kit (Milteny). CD4⁺ T cells were treated with recombinant IL-2. Matured monocyte derived macrophages were counted by 0.4% Trypan blue staining. Live macrophages (1×10⁵) were seeded per well in a 48-well plate and stimulated with 2.5 μg/ml E2 WT or E2 DM for 24 hrs. Live T cells (2.5×10⁴) and macrophages (1×10⁵) were co-cultured for 4 days. On day 5, cells were harvested and stained with CD4-FITC, CXCR3-PE and CCR4-APC antibodies (Biolegend) for FACS analysis.

T-cell activation marker analysis: Isolated CD4⁺T cells from healthy donors were incubated with E2 WT or E2 DM in the presence of 5 μg/ml human anti-CD3 antibody and 1 μg/ml human anti-CD28 antibody (Biolegend) for 2 hrs. Cells were harvested and stained with CD69-FITC and CD25-PE (Biolegend) for FACS analysis. Ab titer and cytokine responses in the spleen cells of immunized mice were observed similar from 1^{st} group of mice after immunization with E2 DM or E2TM.

Production of the mRNA encoding the Luciferase, and modified HCV protein: Codon optimized Luciferase and HCV E2 specific sequence were cloned into an mRNA production plasmid (optimized 3′ and 5′ UTR and containing a polyA tail), in vitro transcribed in the presence of N1-methylpseudouridine modified nucleoside (N1mψ, modified), co-transcriptionally capped using the CleanCap™ technology (TriLink) and cellulose purified (Baiersdörfer et al., 2019; Alameh et al., 2021) to remove double stranded RNA. Purified mRNA was ethanol precipitated, washed, re-suspended in nuclease-free water, subjected to quality control (electrophoresis, dot blot, endotoxin determination using the colorimetric LAL assay and transfection into human DCs), and stored at −20° C. until use.

Production of mRNA-LNP vaccines: mRNA loaded LNPs, or vaccines, were formulated using a total lipid concentration of 40 mM as previously described (Alameh et al., 2021). The ethanolic lipid mixture comprising ionizable cationic lipid, phosphatidylcholine, cholesterol and polyethylene glycol-lipid was rapidly mixed with an aqueous solution containing cellulose-purified N1-mΨ in vitro transcribed mRNAs. The LNP formulation used in this study is proprietary to Acuitas Therapeutics; the proprietary lipid and LNP composition are described in U.S. Pat. No. 10,221,127. RNA-loaded particles were characterized for their size, surface charge, encapsulation efficiency and endotoxin content and subsequently stored at −80° C. at an RNA concentration of 1 μg/μL (in the case of loaded particles) and total lipid concentration of 30 μg/μL-1 (both loaded and empty particles). The mean hydrodynamic diameter of mRNA-LNPs was ˜80 nm with a polydispersity index of 0.02-0.06 and an encapsulation efficiency of ˜95%. Two or three batches from each mRNA-LNP formulations were used in these studies.

E2-mRNA-lipid nanoparticle (LNP) vaccine immunization and protection against recombinant vv/HCV₁₋₉₆₇: Balb/c mice (Jackson Lab) were divided into 3 groups (5 mice per group) and immunized intramuscularly with 10 μg of vaccine antigen twice at 2-week intervals. Test bleeds (3 days before immunization and 3 days before challenge infection with recombinant VV) from mice were analyzed immediately or stored frozen for future use. Immunized mice were challenged IP with live recombinant vv/HCV₁₋₉₆₇ (Ray et al., JVI, 68, 4420-4426, 1994) and sacrificed 4 days after challenge infection for collection of blood, spleen and ovaries and stored at −80° C. until further analyses. Ovaries were homogenized with a motor operated disposable homogenizer, centrifuged for clarity and used for vaccinia virus recovery by plaque assay on BSC-40 cells. Plaques were stained with crystal violet (1% in water: ethanol (60:40) and read.

Statistical analysis: Student's two-tailed paired t-test or unpaired t-test was used to determine the difference within the groups studied. One-way ANNOVA was used to compare between the groups. All the data are expressed as Mean±SE (standard error of the mean), and p<0.05 was considered statistically significant.

### Example 2

### Results

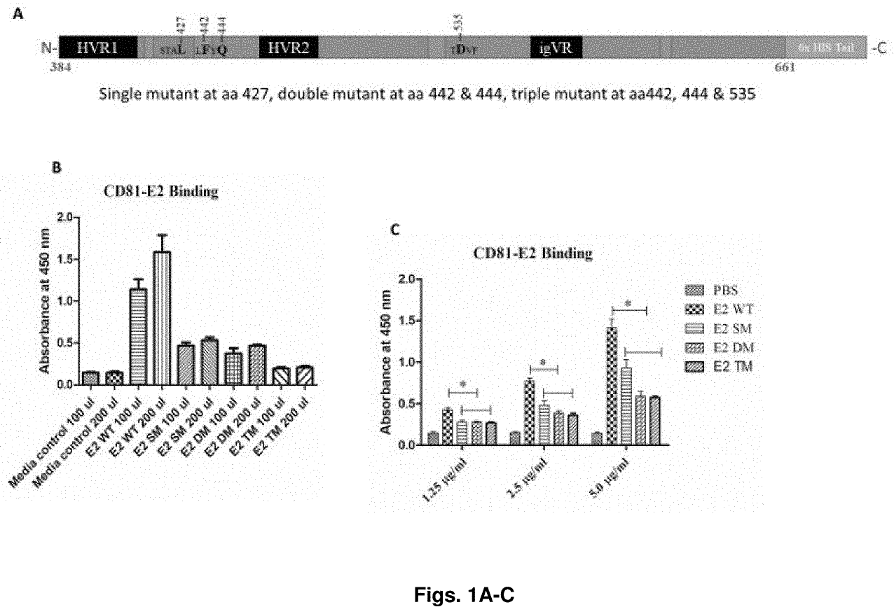

HCV sE2 mutation reduces CD81 binding. Based on available information regarding E2 interaction with the cell surface receptor, CD81, the inventors performed specific mutations (FIG. 1A) and analyzed for ELISA binding. Mutation in F442A did not severely affect the binding of neutralizing antibody to E2 antigen, but it led to nearly 90% reduction in CD81 binding (Harman et al., 2015). Mutation based on modeling information in the literature (Kong et al., 2013) at L427 to Y retains 1% CD81 binding as compared to wild-type E2 in ELISA. Here, the inventors used recombinant affinity purified hexa-histidine tagged E2 protein or mutants from genotype 1a encoding amino acid residues 384-661 representing the ectodomain expressed in HEK293 cells for comparison of immune responses from sE2 and mutated sE2.

The inventors observed binding of mutant secretory sE2 in culture fluid generated from transfected sE2, and sE2 mutant proteins from HEK293 cells (FIG. 1A) when tested against CD81 coated on ELISA plate. Results are shown from 3 independent experiments (FIG. 1B). Each mutant protein exhibited a significant reduction in binding with the CD81 protein as compared to the wild-type E2 sequence.

Introduction of specific amino acids changes in the above-mentioned positions were previously found to abrogate the binding of these mutant proteins to CD81 (Kong, 2013). The E2 single and double mutant used here retained a similar ELISA binding activity with a panel of HCV E2 specific human monoclonal antibodies, CBH2, CBH7, CBH4G, HC-1, HC-33.1, and HC-84, in comparison to the WTE2 protein. As shown in FIG. 1B, the introduction of mutations significantly reduced the CD81 binding ability of the sE2 mutants when compared to wild-type sE2 (p<0.05). Introduction of double mutation (442/444) resulted in a higher reduction in CD81 binding compared to single mutation ((FIG. 1C). This indicates that purification of wild-type sE2 and the mutant sE2 protein sequences retain binding property for use in subsequent experiments.

Inhibition of E2 mutant binding with CD81 skews IL-10/IL-12 response in human macrophage cell line. Both macrophages and DCs play an important role in immune surveillance. The inventors have previously shown that HCV E2 significantly inhibits macrophage polarization toward the M1 phenotype, and antigen presenting DC maturation (Saito et al., 2008; Ray, 2010; Kwon et al., 2019). Therefore, ablating E2 interactions with CD81 will likely have a positive effect for antigen presentation, DC function, and effector B- and T-cell regulation to improve HCV vaccine efficacy.

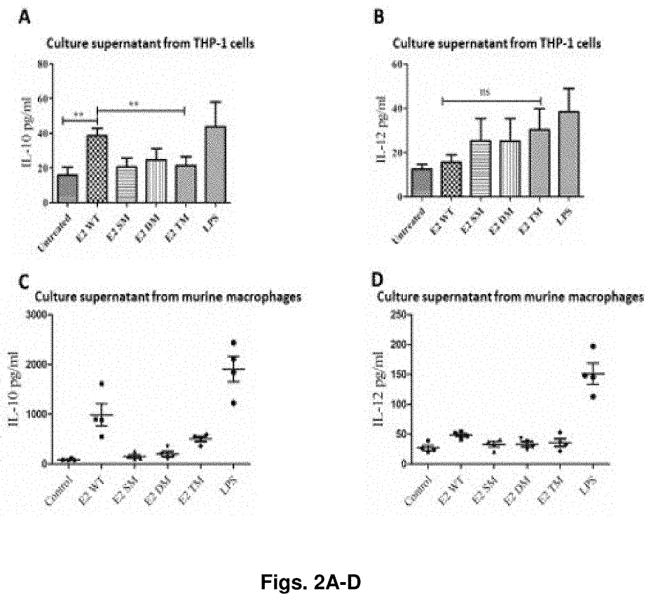

HCV E2 binds to its cognate receptor, CD81, on macrophages and induces the production of the immune suppressive cytokine, IL-10 (Kwon et al., 2019). The inventors hypothesized that a loss of CD81-E2 binding on macrophages would reduce the production of IL-10 by macrophages. Initially the inventors tested his hypothesis in a human monocyte model cell line, THP-1. The inventors stimulated human THP-1 derived macrophages in the presence or absence of wild-type HCV E2 (E2 WT) or single mutant E2 (E2 SM), or double mutant E2 (E2 DM) in vitro. A significantly increased production of IL-10 from THP-1 derived macrophages in wildtype sE2 compared to sE2 mutants and unstimulated control were observed (FIG. 2A). In contrast, sE2 mutants induced an increased IL-12 production compared to wild-type sE2 and unstimulated control (FIG. 2B). The inventors extended sE2 mutant analysis by incubation with mouse peritoneal exudate cells (PEC). In contrast to THP-1, there was a significant difference in IL-10 from PEC in response to E2 stimulation (FIGS. 2C and 2D). The large extracellular loop (LEL) of human CD81 is primarily responsible for CD81-E2 interaction, the mouse has altered amino acids in the LEL region which contribute to the reduced binding affinity to CD81. The present observation strengthens the inventor's hypothesis that binding of CD81-E2 results in the production of IL-10 and loss of this binding results in reduced IL-10 production.

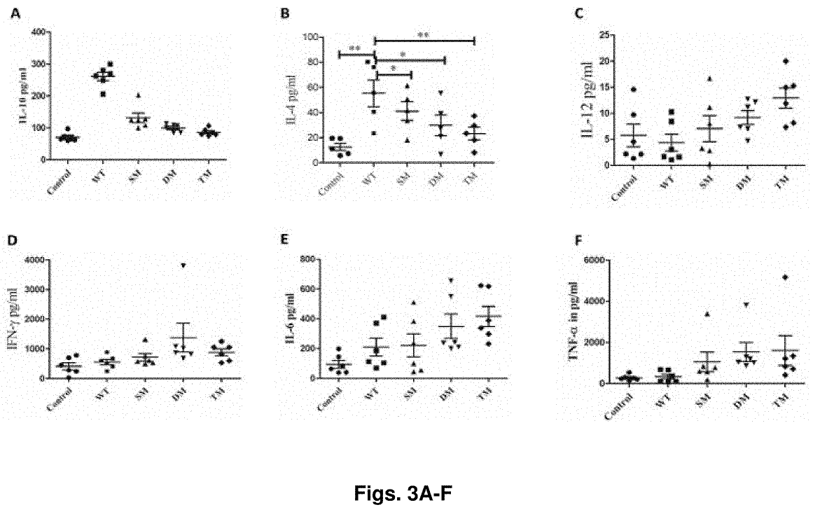

Inhibition of E2-CD81 binding induces pro-inflammatory cytokine production by human monocyte derived macrophages. The inventors tested the immune stimulatory effect of E2 mutant on human monocyte derived macrophages in the subsequent experiment. WT E2 induced significantly higher IL-10 production from macrophages compared to the mutant E2s (<0.01) (FIGS. 3A-F). Within the mutant group, the E2 single mutant produced more IL-10 as compared to the double mutant.

The presence of IL-10 has an inhibitory effect on the production of IFN-γ and suppression of IFN-γ induced genes (Kane, 2001). The inventors measured the production of pro-inflammatory cytokines in the culture supernatant of E2 exposed macrophages. In contrast to IL-10 production, the inventors observed a significant induction of pro-inflammatory cytokines from E2 mutants as compared to wild-type E2 and unstimulated control macrophages (FIGS. 3A-F). Among the tested pro-inflammatory cytokines, IL-12 production was consistently higher in macrophages exposed to mutant E2 protein (FIG. 3C). Production of IL-6 was significantly higher in E2 stimulated groups compared to unstimulated control. Within treatment group, E2 double mutant (DM) produced significantly higher IL-6 compared to single mutant and wild-type E2 (FIG. 3E). The inventors also observed significantly higher generation of TNF-α from E2 mutants in comparison to the wild-type E2 and unstimulated control macrophages (FIG. 3F). Similarly, the inventors found higher production of IFN-γ from double mutant treated cells when compared to other treatments that was approximately 2-fold higher than unstimulated control. The cytokine production responses corroborated with the inventor's CD81-E2 binding results.

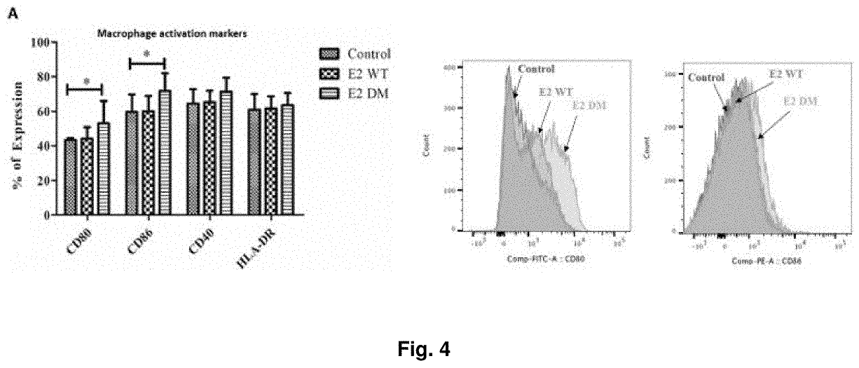

HCV E2 mutant induces monocyte derived M1 macrophage activation markers. Macrophages are sentinels of the immune system found in all tissues and involved in modulation of adaptive immune response starting from pathogen recognition, through antigen processing and presentation to cytokines and activation of other effector cells. M1 and M2 plasticity depends on the cytokine cues present in the extracellular milieu (Stoger, et al. 2012). IFN-γ and TNF-α give rise to classically activated macrophages. M1 activation is associated with STAT1 activation and M2 activation associates with STAT3 activity (Martinez 2014). The E2 mutant showed induction of STAT, whereas wild-type E2 induced STAT3 as previously observed (data not shown). In the previous experiments, the inventors also observed a significant induction of pro-inflammatory cytokines from E2 DM protein compared to unstimulated control and E2 WT stimulated macrophages (FIGS. 3A-F and FIG. 4A).

The M1 macrophages are characterized by high IL-12 and low IL-10 expression, and express high levels of co-stimulatory molecules CD80 and CD86 (Bertani, 2017). These co-stimulatory molecules are expressed on antigen presenting cells and are required for providing necessary co-stimulatory signals for complete activation and survival of T-helper cells (Wang, 2019). In the subsequent experiment, the inventors tested the ability of E2 WT and E2 DM proteins for induction of co-stimulatory molecules CD40, CD80, CD86 and HLA-DR in macrophages. The inventors cultured monocyte derived macrophages from healthy donors in the presence or absence or E2 WT or E2 DM for 24 hrs and measured the expression of co-stimulatory molecules by flow cytometry. A significant increase in expression of CD80 and CD86 in E2 DM stimulated macrophages (<0.05) compared to unstimulated and E2 WT macrophages were observed (FIG. 4A). However, the inventors could not find a difference in the expression of CD40 and HLADR expression between the treatment sets. Taken together, the cytokine profile and enhanced expression of CD80/CD86 indicate that unlike the wild-type E2 antigen, the presence of E2DM induces a profile in macrophages associated with the classical M1 phenotype. Therefore, the inventors took the initial findings into consideration to further explore the immune responses to E2DM as compared to native E2 sequences.

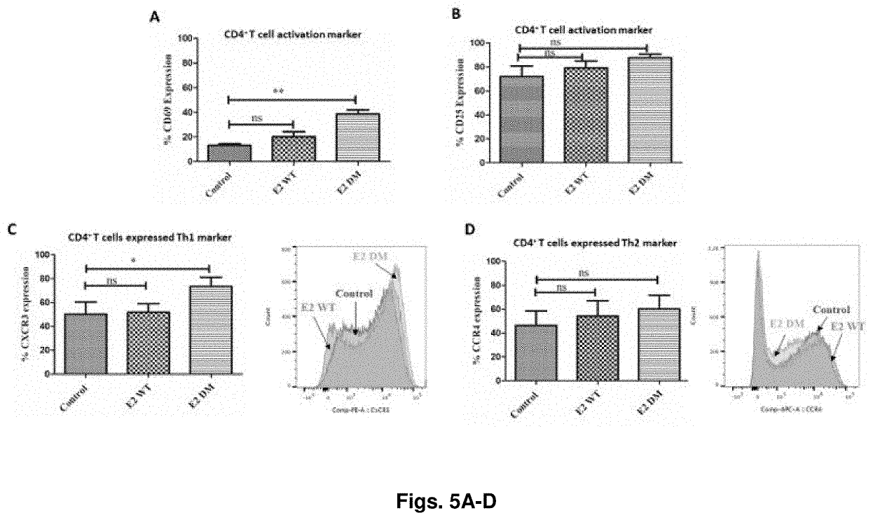

E2 DM activated macrophages promote CD4⁺T cell differentiation into Th1 type. Differentiation of naive T cells into Th1 effector type is critically required to induce a protective immune response against intracellular pathogens (Ref). Polarization of Th0 into Th1 or Th2 type effectors depends on the type and concentration of cytokines present at the immune synapse, the strength of T cell receptor-mediated signals and the activation state of the antigen presenting cells. As E2 DM activates the macrophages towards M1 type, and induces the production of pro-inflammatory cytokines, the inventors hypothesized that these activated macrophages could polarize the T cells towards Th1 type effector cells. To test his hypothesis, the inventors activated the macrophages in the presence or absence of E2 WT or E2 DM for 24 hrs. and co-cultured the autologous CD⁺T cells with the E2 WT or E2 DM activated macrophages. As shown (FIG. 5A), the inventors found a significant increase in the Th1 marker; CXCR3, on CD4⁺T cells co-cultured with E2 DM activated macrophages compared to unactivated or E2 WT activated macrophages. However, the inventors could not find any significant difference (p>0.05) in the expression of the Th2 marker CCR4 on CD4⁺T cells between the different treatment groups (FIG. 5B). Further, the inventors analyzed the expression of the early T cell activation marker. CD69 surface expression correlates with differentiation of CD4⁺T cells into Th1 effector cells (Dorfman 2002). The inventors observed an increased expression of the CD69 activation marker on both E2 WT and E2 DM compared to untreated CD4⁺T cells. However, expression of CD69 on E2 DM treated T cells is significantly (p<0.005) higher compared to E2 WT treated T cells (FIG. 5C). The inventors did not detect a significant change in surface CD25 expression in cells co-cultured with sE2 or E2DM treated macrophages after 24 hours (FIG. 5D).

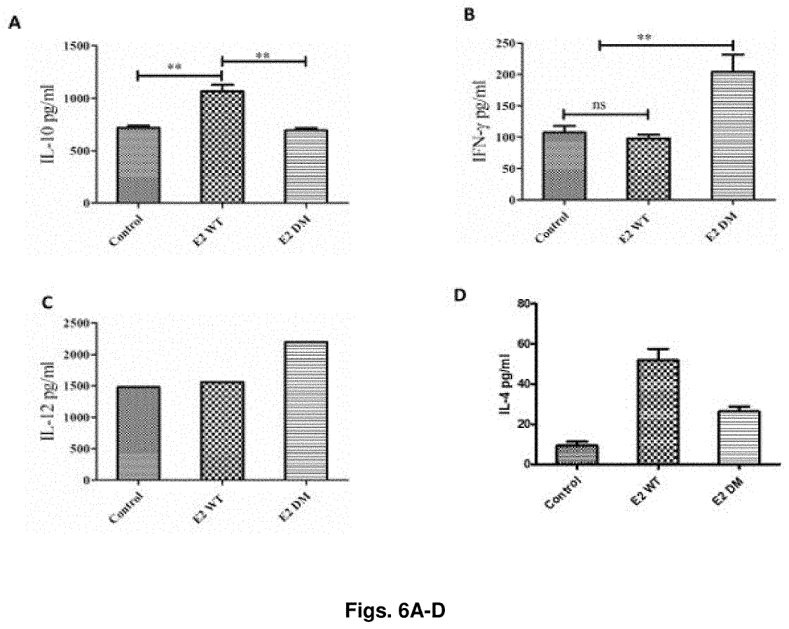

Immunization of mice with E2DM mRNA-LNP vaccine induces pro-inflammatory cytokines. The inventors tested immune stimulatory effect of the E2 mutants in mice immunized with E1-sE2 or E1-E2 DM. sE1-E2 construct induced significantly higher IL-10 production in serum compared to that of E2 mutant vaccine preparation and was highly significant compared to unstimulated control (<0.01) (FIG. 6A). This observation supports the inventor's previous report that the ablation of CD81-E2 interaction on macrophages reduces E2 induced IL-10 production (Kwon et al., 2019).

IL-10 has an inhibitory effect on IFN-γ production and suppression of IFN-γ induced genes (Kane, 2001). In contrast to IL-10 production, the inventors observed a significant induction of IFN-γ in the E1-E2DM vaccinated mice as compared to wild-type E2 and unstimulated control macrophages (FIG. 6B). Among the tested pro-inflammatory cytokines, IL-12 production was consistent across the immunized mice (FIG. 6C). Within treatment groups, E1-E2 DM produced significantly less IL-4 compared to wild-type E1-E2 (FIG. 6D). Taken together, immunization of mice with E1-E2DM mRNA vaccine reversed the IL-10/IL-12 secretion ratio and led to enhanced serum IFN-γ levels.

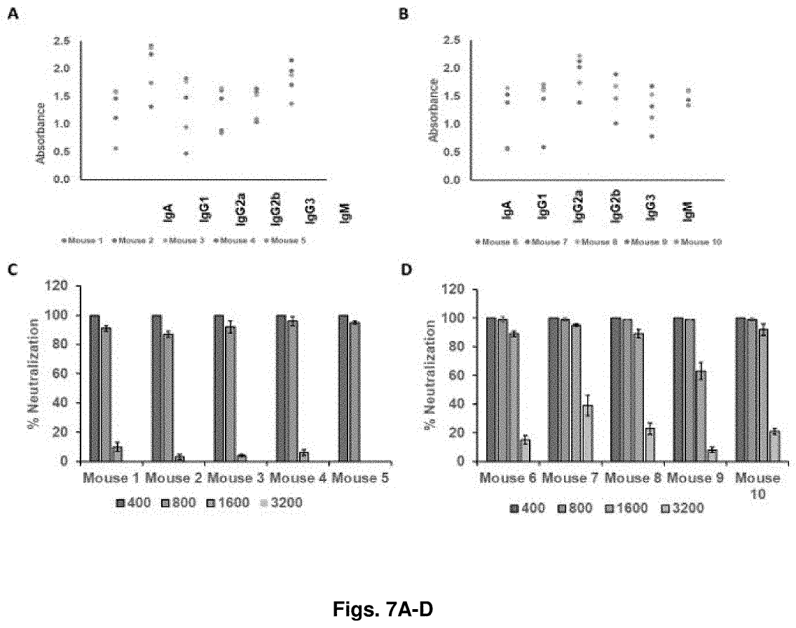

Qualitative nature of antibody improved from E1-E2DM-mRNA vaccine in mice. Antibody isotype switching occurs post antigen exposure as a switch from IgM to other Ig isotypes. Th1 subtype of CD4⁺T cells secrete IFNγ, which induces an isotype switch in B cells to IgG2a secretion, whereas Th2 cells are associated with enhanced IgG1 production. Here, mice immunized with the E1sE2-mRNA vaccine preparation exhibited pronounced IgG1 in serum. In contrast, a distinct skew in the isotype of the E1-E2DM mRNA immunized mice was apparent towards IgG2a production (FIG. 7A). HCV-lentiviral pseudotype system may be used to analyze relative neutralization between these two immunogens. Serum recovered from mice immunized with the E1-E2DM-m-RNA preparation displayed a minimum 2-fold enhancement in neutralization efficacy on HCVpp, as compared to the E1-sE2 immunized mice (FIG. 7B).

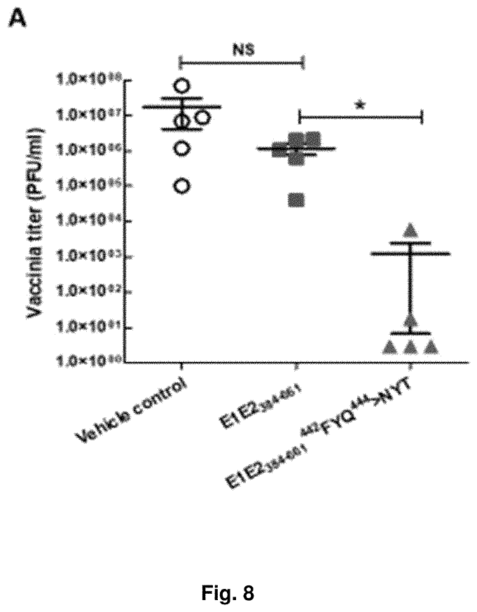

E1-E2DM mRNA vaccine enhances protective immune response to challenge infection with surrogate recombinant vaccinia virus. E1-E2DM mRNA immunized mice were sacrificed after 4 days of challenge infection with surrogate rVV E1/E2/NS2ₐₐ₁₃₄₋₉₆₆. Ovary from experimental animals were homogenized in 300 ul medium, freeze-thawed 3 times and centrifuged to pellet cell debris. Clear supernatant was serially 10-fold diluted and plaque assayed on BSC40 cell monolayer. Plaques were stained after 3 days with 1% crystal violet and counted. Interestingly, vehicle control or wild-type E1-E2mRNA vaccinated mice failed to show a statistically different level in virus recovery by plaque assay (FIG. 8A). In contrast, the E1-E2 DM mRNA vaccine displayed a significant reduction (≥4 log) in virus recovery from the ovary of challenged mice. Two out of 5 mice showed minimal virus recovery, while the remaining 3 displayed an undetected virus titer (FIG. 8A). In conclusion, the E1-E2DMmRNA vaccine preparation induced a significantly improved protective response in immunized animals.

### Example 3

### Discussion

Purified HCV E1/E2 Env GP together with MF59 in a safety and immunogenicity vaccine trial in humans suggested induction of a weak immune response (Frey et al., 2010). MF59 was used in the vaccine trial to facilitate a Th1 cytokine profile and induction of CD4⁺ memory and cytotoxic T-lymphocyte responses. However, the vaccinated volunteers displayed induction of IL-10, with minimal increase in IL-12 expression. The inventor's in vitro study suggested that HCV E2 induced IL-10 in human PBMC derived macrophages, with minimal IL-12 production, and M2 phenotype polarization of macrophages. Macrophages play a crucial role in antigen presentation, and in the interaction between innate and adaptive immunity. M1 macrophages promote Th1 response and possess antiviral activity, while M2 macrophages are involved in the promotion of a Th2 response, and immune tolerance (Mantovani et al., 2011). Here, the inventors examined whether the soluble membrane proximal external region of E2 may be modified as an antigen to produce an improved immunogenic profile for HCV vaccine development. However, the inventors did not analyze at this time the induction of broadly neutralizing antibodies or cross-HCV genotype protection, and duration of protection. These issues will soon be investigated as a follow-up of this study. Protective cytokine and other immune parameters (including B-cells) help in protection from challenge surrogate virus infection. The precise mechanisms for protection from vaccinia infection remain unknown, although CD4⁺T cell-dependent anti-virus Ab production plays an important role in VV clearance (Rong et al., 2004).

Cytokines play an important role in the homeostasis of the immune system, attributing to the development of subsequent resistance or susceptibility to disease (Ribeiro-de-Jesus 1998, Costa-Pereira, 2015). IL-10 is a pleiotropic master regulatory cytokine produced by various immune cells; primarily macrophages, dendritic cells and T helper cells. IL-10 critically associates with the persistence of viruses by suppressing cell mediated immunity and represents a major immunosuppressive cytokine. The role of IL-10 is to limit the extent of the activation of both the innate and adaptive immune cells to maintain a homeostatic state. IL-10 regulates growth and/or differentiation of B cells, NK cells, cytotoxic and helper T cells, mast cells, granulocytes, dendritic cells, keratinocytes, and endothelial cells (Moore et al., 2001). On the other hand, IL-12 orchestrates resistance against infectious diseases by macrophage activation and induces IFN-γ production, effective antiviral response, and helps in eliminating intracellular pathogens (Murray & Wynn, 2011). IL-12 and IFN-γ work concomitantly in the development of Th1 immune response against viral infections (Ref). Exposure of macrophages to IL-10 suppresses IFN-γ induced genes and prevents them from responding to IFN-γ (Ito, S 1999; Kane, 2001). Thus, cytokine produced from infection influences the course of disease (O'Garra, 1994; Ettinger, 2008). An early protective Th1 response favoring IFN-γ production in the presence of IL-12 would be beneficial for a host to prevent virus infection, immunization of HCVE2 mutant and resistance to recombinant VV E1/E2/NS2ₐₐ₁₃₄₋₉₆₆ challenge infection using E1-E2DM-mRNA-LNP supports the use as HCV vaccine. In chronically HCV infected patients, T-cells are inefficient and defective in producing IFN-γ (Semmo et al., 2007). The inventors have shown that interfering with HCV E2-CD81 mediated immune regulation may lead to enhanced immunogenicity for effective vaccine associated protection against infection. The use of E2DM-mRNA vaccine will be most amenable approach for multigenotype based HCV vaccine. Skewing towards Th1 response resulting from disruption of E2-CD81 binding will be appropriate for selection of E2 mutant as candidate for HCV vaccine antigen.

HCV induces interleukin-10 (IL-10) in monocyte-derived DCs and inhibits DC-mediated antigen-specific T-cell activation. IL-12 activates natural killer (NK) cells and induces differentiation of naïve CD4⁺T lymphocytes to become interferon-gamma (IFN-γ)-producing T helper 1 (Th1) effectors in cell-mediated immune responses to intracellular pathogens (reviewed in Ma et al., 2015; PMCID: PMC4754024). IFN-γ priming is a positive feedback mechanism for more robust IL-12 production in certain immune responses. Th1 lymphocytes are initially activated by antigen presenting cell (APC)-derived IL-12 upon pathogen infection.

Individuals who spontaneously clear HCV display broad CD4⁺T-cell responses, stronger T-cell proliferation, higher IL-2, IFN-γ, and TNF-α production than individuals who develop chronic infection dominated by Tregs and IL-10 production (Diepolder et al., 1995; Missale et al., 1996; Urbani et al., 2006; Smyk-Pearson et al., 2008; Kared et al., 2013; Keoshkerian et al., 2016). Strong HCV-specific CD8⁺T-cell responses are generated, and HCV-specific memory T cells persist after recovery from infection (Takaki et al., 2000; Lechner et al., 2000; Shata et al., 2002; Lauer, 2013).

A recent report (Cox et al., 2021) suggested that an HCV T-cell based vaccine regimen encoding virus non-structural NS proteins generated HCV-specific T-cell responses, and lowered the peak HCV RNA level, but did not prevent chronic HCV infection.

We have shown that cells endogenously expressing HCV proteins have perturbed HLA-DR cell surface expression (Saito et al., 2008), associated with the negative regulation of HLA-DR promoters at the transcriptional level. HCV induces interleukin-10 (IL-10) in monocyte-derived DCs and inhibits DC-mediated antigen-specific T-cell activation. HCV E2 induces immune regulatory cytokine IL-10 and CD163 protein expression in primary macrophages (Kwon et al., 2019), and inhibits the function of T-, B- and NK-cells through binding with CD81 (Wack et al., 2001; Crotta et al., 2002; (Rosa et al., 2005; Crotta et al., 2006). HCV-specific CD4⁺T-cell immune deficiency may act as a primary cause of CD8⁺T-cell functional exhaustion (Grakoui et al., 2003; Lauer et al., 2013). The results from this study provide strong important information for the use of a modified HCV E2 antigen in a candidate vaccine and in uncovering mRNA as an ideal vaccine platform for induction of robust protective immune response with the potential to provide an effective vaccine efficacy. In this study, the use of modified E2 combined with HCV E1 on a mRNA-LNP platform induced improved immunity and led to significant neutralization of surrogate vaccinia-HCV infection in a mouse model. The inventors also need to evaluate the use of E2 mutant as therapeutic vaccine. The results from such a study will considerably advance highly effective vaccine development against persistent hepatotropic HCV cross-genotype specific infection using the mRNA-LNP vaccine approach.

### Example 4

### Materials and Methods

Generation of mRNA-LNP vaccines encoding HCV envelop glycoproteins. Codon optimized luciferase, and HCV (genotype 1a/H77C) envelop glycoproteins sE1 (aa 250-351), sE2 (aa 383-661) and modified sE2_{F442NYT} specific sequence were constructed. HCV sE2 and sE2_{F442NYT} protein samples were examined and compared for the inserted glycosylation site at F442 after PNGaseF treatment at the Washington University Proteomics Shared Resource (WU-PSR), St. Louis. The glycosylation site at the residue F442 of modified sE2_{F442NYT} protein was observed following constitutive deamidation of that residue and variable deamidation of other asparagine residues in that sequence. The mRNAs from HCV sE1, sE2, or sE2_{F442NYT} were generated, purified, and loaded as LNPs candidate vaccine formulated for the study as previously described (Vijayamahantesh et al., 2022). mRNA loaded LNPs were formulated using a total lipid concentration of 40 mM.

Immunization of mRNA-LNP vaccine and challenge infection using recombinant vaccinia virus. BALB/c mice (Jackson Lab) were divided into five groups (5 mice per group) and each group of mice were immunized intra-muscularly with 10 μg mRNA-LNP candidate vaccine as sE1/sE2, sE1, sE2, sE2_{F442NYT}, sE1/sE2_{F442NYT}, or vehicle control twice at 2-week intervals. HCV vaccinia challenge as a surrogate model is helpful in analyzing protective response to challenge infection (Vijayamahantesh et al., 2022; Crawford et al., 2020; Olivera et al., 2020; Murata et al., 2003). Immunized mice were challenged intra-peritoneally with live recombinant vaccinia virus expressing HCV E1-E2-NS2₁₃₄₋₉₆₆ or HCVE2-NS2-NS3₃₆₄₋₁₆₁₉ (genotype 1a/H77C) and sacrificed 4 days after challenge infection for collection of the ovaries for further analysis. Ovaries were homogenized, freeze-thawed three times and centrifuged. Clear supernatant was serially diluted for measuring vaccinia virus titer by plaque forming assay on BSC-40 cell monolayer. After 3 days, plaques were stained with 1% crystal violet and counted.

Ethical statement. All animal experiments were conducted in accordance with the relevant local, state, and federal regulations. All studies were approved by the Saint Louis University Institutional Animal Care and Use Committee (IACUC).

Cytokine quantification. Test bleeds (3 days before immunization and 3 days before challenge infection) from experimental mice were analyzed. The serum cytokines, IL-2 (Sigma), IFN-γ (R&D Systems), IL-4 (Invitrogen) and IL-10 (Invitrogen) were measured from the immunized mice by ELISA using commercially available kits following the manufacturer's instruction. Similarly, mouse IL-2, IFN-γ and Granzyme B (Sigma) were quantified from ovary homogenates of the mice after challenge infection with recombinant vaccinia virus.

HCV pseudoparticle neutralization assay. HCV pseudo particles (HCVpp) were generated by co-transfection of HEK293T cells with the HDM-Hgpm2-pRC-CMV-Rev1b-HDM-tat1b packaging vector, Luciferase-IRES-ZsGreen plasmid (BEI Resources), and plasmid expressing HCV (H77C) E1E2 using Lipofectamine 3000 from ThermoFisher (Crawford et al., 2020). Supernatants containing HCVpp were harvested 72 h post-transfection and filtered through a 0.45-μm pore size membrane. For neutralization testing of HCVpp, 1.5×10⁴ Huh7.5 cells per well were plated in 24-well tissue culture plate and incubated overnight at 37° C. The following day, HCVpp were mixed with or without different serial dilutions of the immunized mouse sera and incubated for 1 h at 37° C. before adding to Huh7.5 cells. After 72 h at 37° C., cells were lysed with cell lysis buffer (Promega) and 100 μl of luciferase substrate (Promega) was added to each well. Luciferase activity was measured in relative luminescence units (RLU) using Glomax luminometer (Promega). Cells were washed, and the inhibitory dilution of neutralization of pseudotype infectivity (IC₅₀) was defined as ≥50% reduction of luciferase activity as previously described⁶. Neutralizing activities were presented using lower and upper bounds (0% and 100% inhibition) as constraints to assist nonlinear curve fitting.

Subclass or isotype specific immunoglobulin response. Nunc MaxiSorp ELISA plates were coated with 1 μg/ml purified HCV E1E2 proteins (Chiron) in 50 μl 0.1 M sodium bicarbonate buffer (pH 7.2) overnight at 4° C. The wells were blocked with 2.5% BSA/PBS blocking buffer for 2 h at 37° C. and washed four times with 0.01% Tween 20 in PBS. Mouse sera were serially diluted in blocking buffer, added to the plate, and incubated overnight at 4° C., followed by washing. Different rabbit HRP-tagged anti-mouse immunoglobulins (subclass: IgG, IgM, IgA; and isotype: IgG1, IgG2a, IgG2b, IgG3) from Bio-Rad were added to the appropriate wells and incubated for 1 h at 37° C., followed by four washes. 100 μl of peroxidase substrate solution was added and the reaction was stopped with 2 M sulfuric acid. The absorbance was measured at 450 nm using an ELISA plate reader (Tecan).

HCV envelop peptide specific reactivity. Peptide specific serum IgG binding was tested using ELISA with 18-mer peptides from E1 (aa 314-331), E2 (aa 404-421) and E2 (aa 429-446) of HCV H77 strain (BEI Resources) representing diverse genotype specific conserved neutralizing epitopes (Tarr et al., 2006; Owsianka et al., 2005; Meunier et al., 2008; Tarr et al., 2012; Wong et al., 2014; Ball et al., 2014). A SARS-CoV-2 fusion domain peptide was used as an irrelevant control. Wells were coated with 500 ng of peptide and ELISA was performed using different dilution of immunized mouse sera as described above.

Statistical analysis. All data were analyzed using GraphPad Prism 7 software. Analysis of data with only two groups was conducted utilizing a one tailed Mann-Whitney U test or unpaired t test. One-way ANOVA with Kruskal-Wallis's test was used for multiple pairwise comparisons between groups. All the data are expressed as Mean±SD (standard deviation of the mean), and p<0.05 was considered statistically significant.

### Example 5

### Results

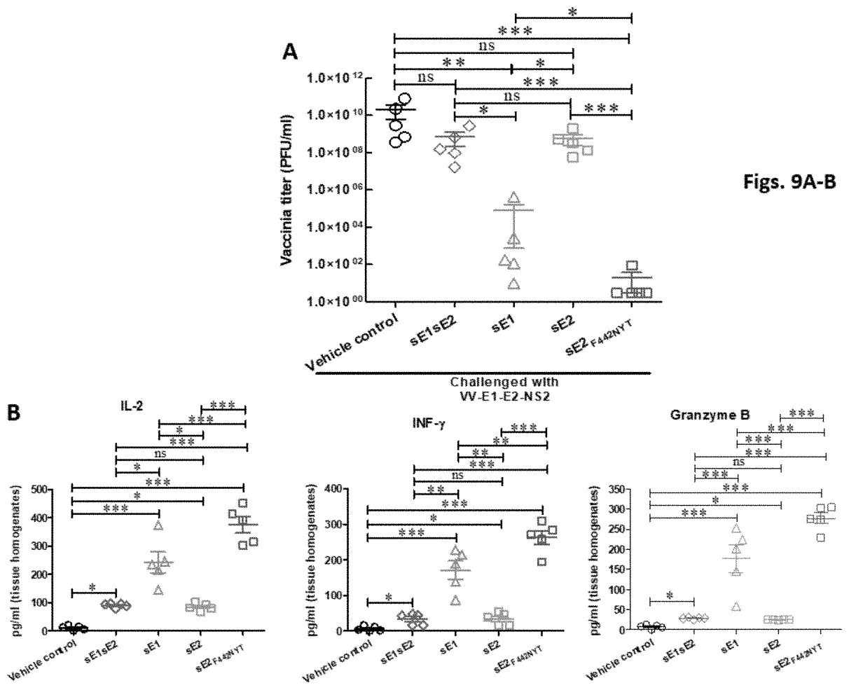

A modified HCV envelope mRNA-LNP vaccine confers protective cellular immunity. The absence of an appropriate small animal model that mimics the natural host for HCV infection is one of the biggest obstacles in evaluating vaccine efficacy. To assess the biological relevance of the protective immunity induced by mRNA-LNP vaccination with different HCV envelope constructs, the inventors utilized a surrogate challenge model of a recombinant vaccinia virus expressing HCV E1-E2-NS2. The recombinant virus was intraperitoneally inoculated (10⁷ pfu in 200 μl) 2 weeks after final mRNA-LNP vaccination of BALB/c mice. Mice were sacrificed 4 days later, and the ovaries were harvested for vaccinia virus plaque recovery. The inventors observed that sE1/sE2 in combination or sE2 alone immunized mice demonstrated ˜1-log reduction (p=0.1348 and p=0.1223) in the mean virus titer in comparison to the mRNA-LNP vehicle control group (FIG. 9A). While four of the five sE2_{F442NYT} immunized mice did not exhibit a detectable virus titer after challenge infection with recombinant vaccinia virus, only one animal had an ˜10-log reduction in virus titer (p=0.0006). Interestingly, the sE1 alone immunized mouse group also had a several folds decrease in viral titer (>4 log, p=0.0097) as compared to the control group (FIG. 9A). Thus, the modified sE2 mRNA-LNP (sE2_{F442NYT}) vaccine preparation induced the strongest protective immune response in the surrogate challenge animals. These results further exhibited that the protective cellular response from vaccination with mRNA-LNPs expressing HCV E1 was greatly reduced in the presence of native HCV E2 mRNA-LNPs.

Earlier studies suggested that a surrogate recombinant vaccinia virus challenge model confers tissue specific cellular immunity. The inventors analyzed the tissue specific level of IL-2 and IFN-γ in the clarified ovary homogenate of the recombinant vaccinia virus challenged mRNA-LNP vaccinated mice and observed a significant increase in IL-2 and IFN-γ (FIG. 9B). Granzyme B, a key cytolytic mediator released from cytotoxic T lymphocytes (CTL) into the virus-infected tissues helps in clearing virus (Trapani & Smyth, 2002). Tissue specific levels of granzyme B correlate as a traditional determinant of CTL mediated activity. Several studies have measured tissue specific granzyme B level along with other cytokines as an indicator of cellular immunity (Salti et al., 2011). Granzyme B expression in the ovary homogenates of sE2_{F442NYT} immunized mice was increased (FIG. 9B). The sE1 alone immunized group showed a substantial expression of IL-2, IFN-γ and granzyme B (FIG. 9B). However, sE1/sE2 in combination or sE2 alone immunized mice exhibited a very low expression level. These results on immune-modulators correlated with detectable vaccinia titer supporting CTL-mediated immunity in this surrogate mouse model. It further identified that sE2 may act to suppress cytokine and cellular immune responses generated by co-expressed molecules; in this instance, the sE1 mRNA-LNP.

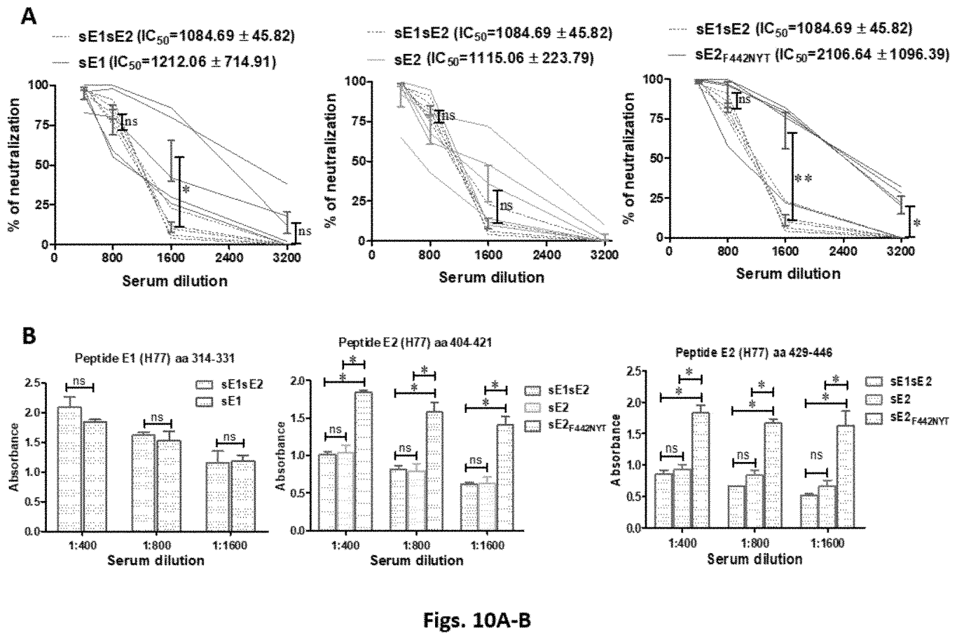

Modified sE2 mRNA-LNP elevates neutralizing antibody response. To understand the comparative effects of antibody neutralization potency of each HCV envelope-mRNA candidate vaccine component, the inventors analyzed HCV-lentiviral pseudotype particle (HCVpp) representing homologous H77C neutralization by serum from individual vaccinated mice. The sE2_{F442NYT} immunized group displayed an almost 2-fold enhancement in neutralization efficacy of HCVpp as compared to the sE1/E2 combined immunization, while vaccination with solitary sE2 did not reflect any significant difference from the combined sE1/sE2 immunization (FIG. 10A). In some cases, sera from sE1 immunized mice exhibited higher neutralization in comparison to the combined sE1/sE2 immunized group (FIG. 10A), but protection was not consistent among the samples.

The inventors extended their study to investigate whether the modified HCV envelope-mRNA candidate vaccine induces antibodies particularly recognizing the conserved E1 or E2 epitope associated with broad neutralization activity. For this, the inventors used one E1 based peptide spanning amino acid region 314-331 and two E2 based peptides spanning amino acids 404-421 and 429-446, responsible for HCV multi-genotype neutralization (Tarr et al., 2006; Owsianka et al., 2005; Meunier et al., 2008; Tarr et al., 2012; Wong et al., 2014; Ball et al., 2014). ELISA was performed by coating these peptides and using serially diluted vaccinated mouse sera. Analyses involving ELISA reactivity using specific peptides identified minimal changes in reactivity to a conserved E1 specific peptide. In contrast, immunization of mice with the sE2_{F442NYT} construct conveyed a significant increase in reactivity to both E2 specific peptides at dilutions shown here (FIG. 10B). Thus, these results suggest that immunization with the modified sE2_{F442NYT} mRNA-LNP vaccine elicits a stronger antibody binding response (4-fold higher), as compared to the unmodified envelope glycoproteins.

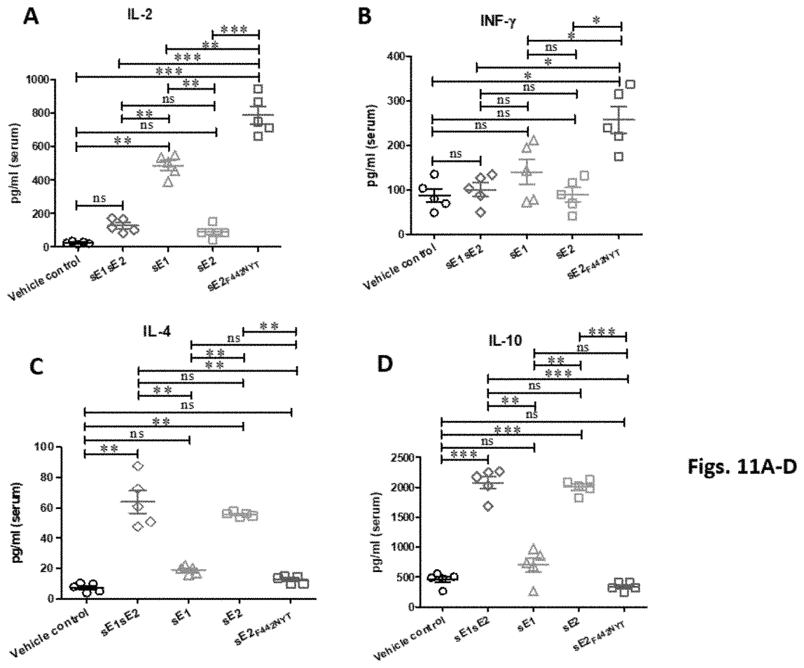

Induction of Th1 specific response in sE2_{F442NYT} vaccinated mouse sera. The inventors further analyzed Th1 and Th2 specific cytokines by ELISA in the serum of HCV envelope-mRNA-LNP vaccinated mice. Th1 specific cytokines, IL-2 and IFN-γ, were significantly elevated in the sE2_{F442NYT} immunized group; whereas, the sE1 alone immunized group showed an enhanced IL-2 and IFN-γ level (Fig. 11. 3A-B). The sE1/E2 in combination or sE2 alone immunized mice had a low level of Th1 specific cytokines. Conversely, the Th2 specific cytokines, IL-4 and IL-10, were increased in sE1/sE2 in combination or sE2 alone immunized mice. However, the sE2_{F442NYT} or sE1 alone immunized group demonstrated a marginal level of IL-4 and IL-10 (FIGS. 11C-D). These results suggested that immunization with sE2 alone induced stronger Th2 specific cytokines while sE1 alone exhibited an opposite effect. Further, combined immunization of sE1/sE2 displayed a cytokine profile similar to that defined by vaccination with sE2 alone, further reinforcing the modulating nature of sE2. On the other hand, sE2_{F442NYT}mRNA-LNP induced Th1 specific cytokines and had a reduced Th2 response in vaccinated mice.

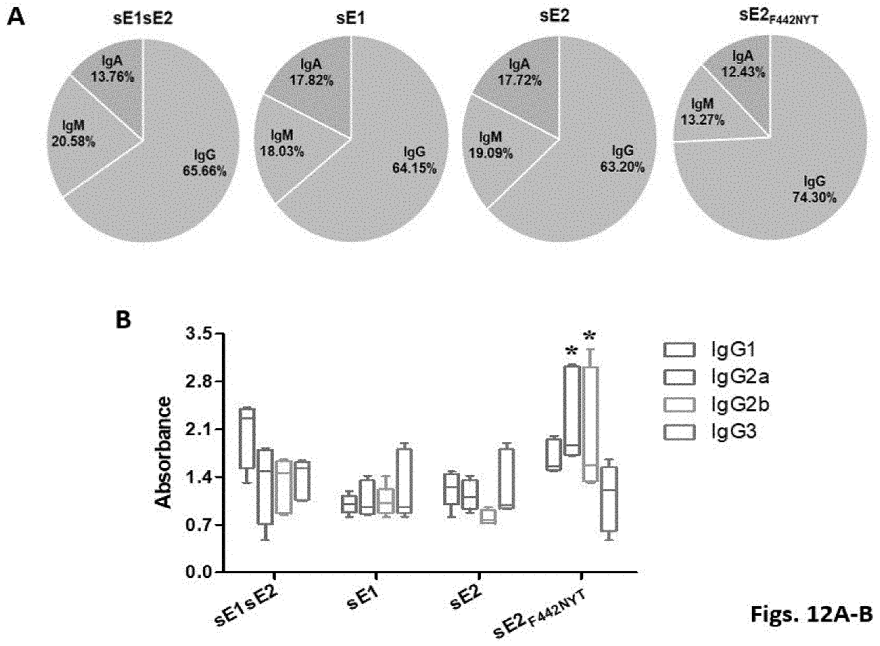

The distribution of antigen specific immunoglobulin subclasses obtained from the serum of HCV envelope-mRNA-LNP vaccinated animals is shown in pie diagrams (FIG. 12A). The inventors observed an increase in total IgG production only in the sE2_{F442NYT} immunized group of mice, whereas the other immunized groups did not show a significant variation in IgG, IgA, or IgM production (FIG. 12A). The inventors also analyzed IgG specific isotype switching in mouse sera after different HCV envelope antigen exposure. A switch from IgG1 to IgG2a and IgG2b was observed in sE2_{F442NYT} immunized mice, and isotype switching was not observed upon immunization with the other HCV specific mRNA-LNPs (FIG. 12B).

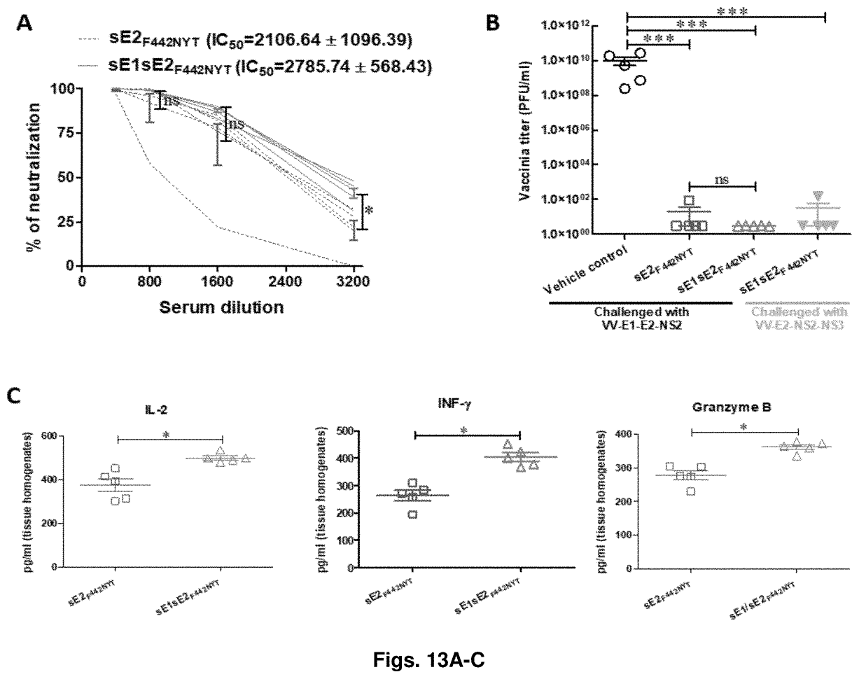

Inclusion of sE1 with sE2_{F442NYT} may be beneficial for HCV vaccine generation. The inventors have identified that the use of sE1 as a vaccine antigen has moderate protective efficacy from antibody neutralizing responses, and significantly induces CTL-mediated immunity. Further, sE2_{F442NYT} mRNA-LNP proves to be a better vaccine candidate among the selected antigens. Next, the inventors examined whether combined immunization of sE1 and sE2_{F442NYT} further increases the protective efficacy of the vaccine preparations. The combined sE1s/E2_{F442NYT} immunization showed ˜10-20% enhancement of HCVpp neutralization potency in comparison to sE2_{F442NYT} immunized mouse serum (FIG. 13A). HCV E1 glycoprotein has distinct pan-genotype neutralizing epitopes. Therefore, the addition of sE1 with sE2_{F442NYT} mRNA-LNP could widen the neutralizing breadth of the vaccine candidate, particularly in the absence of the suppressing nature of sE2. In the surrogate challenge model, the inventors observed that four of the five sE2_{F442NYT} immunized mice did not exhibit a detectable recombinant vaccinia virus titer, and the inventors did not recover virus from five combined sE1/sE2_{F442NYT} immunized mice (FIG. 13B). On the other hand, the presence of sE1 with sE2_{F442NYT} as vaccine antigen did not impair the overall protection level when challenged with another recombinant vaccinia virus expressing HCV E2-NS2-NS3 (FIG. 13B). On the other hand, a significant increase in the IL-2, IFN-γ and granzyme B level in the ovary homogenates of combined sE1/sE2_{F442NYT} vaccinated mice was observed when compared to sE2_{F442NYT} mRNA-LNP immunized group (FIG. 13C) and seems to indicate a better cellular immune response for the combination of E1 and the E2 mutant relative to the E2 mutant alone. Thus, these results indicated inclusion of sE1 with sE2_{F442NYT} for vaccination may provide protection benefit.

### Example 6

### Discussion

Observations from this study suggest that the modified sE2 mRNA-LNP (sE2_{F442NYT}) as a candidate vaccine induced a strong protective cellular immune response in surrogate mouse model. Further, an enhanced neutralizing antibody response was observed using a homologous HCVpp model. Importantly, a protective immune response generated by sE1 immunization is suppressed in the presence of sE2 introduced as mRNA-LNP vaccine. The suppressive effect of sE2 may be due to the generation of an enhanced anti-inflammatory cytokine response. Immunization with sE2 alone induced stronger Th2 specific cytokines, while sE1 exhibited an opposite effect. Further, combined immunization of sE1/sE2 displayed a cytokine profile similar to that of vaccination with sE2 alone. On the other hand, sE2_{F442NYT} mRNA-LNP induced Th1 specific cytokines in vaccinated mice. Granzyme B expression in the ovary homogenates from sE2_{F442NYT} immunized mice was increased. However, sE1/sE2 combination or sE2 alone immunized mice exhibited very low granzyme B expression, while sE1 alone immunization showed a substantial expression of IL-2, IFN-γ and granzyme B. Although, sE2_{F442NYT} mRNA-LNP candidate vaccine induced a stronger binding and neutralizing antibody response, T-cell based immune responses appeared to play a more important role for protection in the experimental surrogate vvHCV challenge model, as recovered viral titers were profoundly reduced in response to sE2_{F442NYT} immunization.

HCV E2 induces IL-10, affects CD8⁺T cell functions, and inhibits granzyme release¹⁸. In contrast, the sE2_{F442NYT} immunized mouse serum had a significantly higher level of the pro-inflammatory cytokines, INF-γ and IL-12, low levels of the anti-inflammatory cytokine IL-10 and immuno-regulatory cytokine IL-4. sE2_{F442NYT} immunization potentiates a strong neutralization effect on HCVpp (IC₅₀=2106.64±1069.39), while sE1 exerts almost similar (IC₅₀=1212.06±714.91) neutralization titer as sE2 immunization did (IC₅₀=1115.06±223.79). Combined immunization of sE1 and sE2_{F442NYT} had a little advantage in neutralizing potency (IC₅₀=2785.74±568.43). These results also indicated a significant increase in serum IL-2, IFN-γ and ovary granzyme B in recombinant vaccinia virus challenged sE2_{F442NYT} immunized mice for functional make-up of effector T-cells. Thus, sE2_{F442NYT} appeared to be a superior antigen to sE2. Further, E1 had protective antigenic properties when evaluated alone. The sE1/sE2_{F442NYT} combination will likely provide a stronger breadth and greater recognition of multiple antigenic sites. An increase in total IgG production was also observed in the sE2_{F442NYT} immunized mice, while other immunized groups did not show a significant variation in IgG, IgA, or IgM level. A switch from IgG1 to IgG2a and IgG2b was observed in sE2_{F442NYT} immunized mice, and isotype switching was not observed upon immunization with other HCV specific mRNA-LNP candidate vaccines. The IgG isotype switching further supports a strong Th1 response as evident from sE2_{F442NYT} mRNA-LNP vaccinated mice.

These results indicate that the use of a soluble wild type E2 could restrict immune response from the mRNA-LNP platform, and may provide limited benefit as a component of a HCV vaccination strategy. However, the use of a modified soluble E2-mRNA-LNP (in this case sE2_{F442NYT}) could contribute toward a strong immune response in a HCV vaccine. The mRNA-LNP vaccine platform would likely offer an excellent opportunity for use in a multi-genotype HCV vaccine to accommodate a number of HCV E2 sequences to induce a broad protective response, if cross genotype neutralization proved to be inefficient. The use of both the E1 and sE2_{F442NYT} mutant (sE2DM) will likely offer a much broader neutralizing epitope specific repertoire of antibodies and T-cell mediated immune responses, allowing for greater vaccine efficacy despite the potential loss of limited antigenic sites/epitopes from the E2_{F442NYT} modification. There is debate regarding the need for conformationally correct E1 and E2 envelope glycoproteins in HCV vaccines. This may be important for further clarification. At this time, the inventors are not sure what optimum conformation and how many neutralizing antigenic sites need to be intact in E2 for a highly effective vaccine. Further the abundance and frequency of the antibodies to these conformational domains in spontaneously clearing humans appear to remain unknown. These results suggest that sE1 vaccination induced higher neutralization titers as compared to sE2 or sE1/sE2 and could also induce a T-cell associated cytokine response for protection against recombinant vv/HCV challenge infection. Protection against challenge infection in a mouse model may likely be contributed to by both CD4⁺ and CD8⁺T cell responses as suggested earlier (Murata et al., 2003). A previous study by the inventors revealed that HCV E2 binding to CD81 tilts immune function towards a Th2 cytokine associated response. This was evident using a mRNA-LNP vaccine platform that normally promotes Th1-biased immune responses (Alameh et al., 2021; Pardi et al., 2018) where the presence of E2 led to a clear shift to a Th2 response.

There are limitations to this study, most notably the lack of an immuno-competent small animal model susceptible to HCV infection. The vaccinia virus challenge model is a surrogate system, not reflective of the actual virus, and protection or clearance from natural HCV infection. Further, this study did not consider in depth heterologous genotype or pan genotype related protection which has been recognized as circulating HCV in the communities in different geographic regions, and this must be addressed. Future studies will focus in extending these findings and determine the nature of protective immune responses for virus clearance in other animal models, including in nonhuman primates. The role of antibodies in virus clearance, memory B- and T-cell responses will be the other obvious aspects to understand from in vivo results. Current studies are ongoing to address some of these difficult but important areas.

Immunization with the sE2_{F442NYT} mRNA-LNP induces a much improved proinflammatory cytokine response, T-cell response, immunoglobulin class switching, neutralizing antibody induction, and a strong protective efficacy against a surrogate vv/HCV challenge infection in mice. This study's limitation includes immunization and infection of only female mice as a model and challenge with a HCV surrogate virus due to constraints with availability of small animals permitting HCV infection for candidate vaccine evaluation. To enhance the protective efficacy of the variable HCV E2, the inventors observed that the use of a relatively conserved region of the E1 ectodomain alone also induces protective T-cell responses. Booster immunization with peptides representing conserved B-cell and T-cell epitopes from multiple HCV genotypes to mRNA-LNP vaccinated mice may also find additional benefits. Thus, these results will considerably advance HCV vaccine development by incorporating sE1/sE2_{F442NYT} in an mRNA-LNP platform for induction of a broad protective immune response. These observations are important and hold a strong potential in initiating new avenues for understanding the immunogenicity of selected antigens for a candidate vaccine in higher primates and humans to accelerate HCV vaccine development.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### VII. REFERENCES

The following references, to the extent that they provide exemplary procedural or other details supplementary to those set forth herein, are specifically incorporated herein by reference.
Alameh et al., Immunity, 54, 2877-2892 (2021).
Baiersörfer et al., Mol Ther Nucleic Acids 2019;15:26-35.
Ball et al., Antiviral Res. 105, 100-111 (2014).
Crawford et al., Viruses. 12, 513 (2020).
Crotta et al., J Exp Med. 7; 195(1):35-41 (2002).
Crotta et al., Eur. J. Immunol. 36, 919-929 (2006).
Flint et al., J. Virol. 73:6235-6244 (1999).
Flint et al., J. Virol. 74:702-709 (2000).
Frey et al., Vaccine, 28, 6367-6373 (2010).
Grakoui et al., Science. 302 (5645):659-662 (2003).
Harman et al. “A view of the E2-CD81 interface at the binding site of a neutralizing antibody against hepatitis C virus.” J. Virol. 89.1 (2015): 492-501.
Kong et al. Science 342 (6162):1090-4 (2013).
Keck et al., Mapping Determinants of Virus Neutralization and Viral Escape for Rational Design of a Hepatitis C Virus Vaccine. Front. Immunol., 31 May 2018.
Kwon et al., Hepatology, 69, 1873-1884 (2019).
Lauer, The Journal of Infectious Diseases, 207, S7-S12 (2013).
Mantovani et al., Eur J Immunol. 41, 2522-2525, (2011).
Meunier et al., J Virol. 82, 966-973 (2008).
Michalak et al., J. Gen. Virol. 78:2299-2306 (1997).
Moore et al., Annu Rev Immunol. 19:683-765 (2001).
Murata et al., Proc. Natl. Acad. Sci. USA. 100, 6753-6758 (2003).
Murray, P. J. & Wynn, T. A. Protective and pathogenic functions of macrophage subsets. Nat Rev Immunol. 11, 723-737,2011.
Page et al., Randomized Trial of a Vaccine Regimen to Prevent Chronic HCV Infection. N Engl J Med 2021; 384:541-9. DOI: 10.1056/NEJMoa2023345.
Pardi et al., J Exp Med. 4; 215(6):1571-1588 (2018).
Park & Rehermann, Immunity. 40: 13-24 (2014).
Pestka et al., Proc. Natl. Acad. Sci., 104 (14) 6025-6030 (2007).
Olivera et al., Arch Virol. 165, 593-607 (2020).
Osburn et al., Hepatology. 59(6): 2140-2151 (2014).
Owsianka et al., J. Virol. 79, 11095-11104 (2005).
Ray et al., J Virol. 68, 4420-4426 (1994).
Ray et al., J Infect Dis. 202, 862-866 (2010).
Domenico et al., PNAS Dec. 20, 2005 102 (51) 18544-18549.
Salti et al., J. Immunol. 187, 6301-6309 (2011).
Tarr et al., Hepatology 43, 592-601 (2006).
Tarr et al., J. Virol., 86, 2739-2749, (2012).
Thimme, Journal of Hepatology. 74, 220-229 (2021).
Trapani & Smyth, Nature Reviews Immunology 2, 735-747 (2002)
Rong et al., J Immunol., 2004; 172:6265-71.
Wack et al., Eur J Immunol. 31(1):166-75 (2001).
Wong et al., J Virol. 88, 14278-14288 (2014).
Vijayamahantesh et al., J. Virol. 96:e0052322 (2022).

1. A method of generating protective immune response in a subject at risk of contracting HCV comprising delivering to said subject a polypeptide comprising residues 384 to 661 of HCV sE2 glycoprotein with one or more substitutions of L427Y, F442N, Q444T and/or D535A as compared to reference sequence SEQ ID NO: 1.

2. The method of claim 1, wherein the only substitution is L427Y.

3. The method of claim 1, wherein the only substitution is F442N.

4. The method of claim 1, wherein the only substitution is Q444T.

5. The method of claim 1, wherein the only substitutions are F442N and Q444T.

6. The method of claim 1, wherein the polypeptide comprises or is limited to 2, 3 or 4 of said substitutions.

7. The method of claim 2, wherein said RNA or DNA sequence is delivered as part of a lipid nanoparticle.

8. The method of claim 1, wherein the subject is infected with HCV determined by diagnostic testing and/or clinical diagnosis for potential use as a therapeutic vaccine.

9. The method of claim 1, wherein the subject has been exposed to HCV but is asymptomatic for potential use as a therapeutic vaccine.

10. The method of claim 1, wherein the subject is neither infected nor has been exposed to HCV for use as a prophylactic vaccine.

11. A hepatitis C virus (HCV) polypeptide comprising residues 384 to 661 of HCV sE2 glycoprotein with one or more substitutions of L427Y, F442N, Q444T and/or D535A as compared to reference sequence SEQ ID NO: 1.

12. The polypeptide of claim 11, wherein the only substitution is L427Y.

13. The polypeptide of claim 11, wherein the only substitution is F442N.

14. The polypeptide of claim 11, wherein the only substitution is Q444T.

15. The polypeptide of claim 11, wherein the polypeptide comprises or is limited to 2, 3 or 4 of said substitutions, such as wherein the substitutions are only F442N and Q444T or only wherein the only substitutions are L427Y, F442N and Q444T.

16. A vaccine formulation comprising a hepatitis C virus (HCV) peptide or polypeptide comprising residues 384 to 661 of HCV sE2 glycoprotein with one or more substitutions of L427Y, F442N, Q444T and/or D535A as compared to reference sequence SEQ ID NO: 1.

17. The vaccine formulation of claim 16, wherein said formulation is lyophilized.

18. The vaccine formulation of claim 16, wherein said formulation is a liquid formulation comprising said peptide or polypeptide in a pharmaceutically acceptable diluent.

19. The vaccine formulation of claim 16, further comprising an adjuvant.

20. The vaccine formulation of claim 16, wherein said formulation is sterile.

21. A vaccine formulation comprising an RNA or DNA encoding hepatitis C virus (HCV) peptide or polypeptide comprising residues 384 to 661 of HCV sE2 glycoprotein with one or more substitutions of L427Y, F442N, Q444T and/or D535A as compared to reference sequence SEQ ID NO: 1.

22. The vaccine formulation of claim 21, wherein said formulation is lyophilized.

23. The vaccine formulation of claim 21, wherein said formulation is a liquid formulation comprising said RNA or DNA in a pharmaceutically acceptable diluent.

24. The vaccine formulation of claim 21, wherein said RNA or DNA is in a lipid nanoparticle.

25. The vaccine formulation of claim 21, wherein said formulation is sterile.
